(19) United States
(12) Patent Application Publication
(10) Pub. No.: US 2022/0382730 A1
(43) Pub. Date: Dec. 1, 2022

(54) IDENTIFICATION OF MATCHED SEGMENTED IN PAIRED DATASETS

(71) Applicant: Ancestry.com DNA, LLC, Lehi, UT (US)

(72) Inventor: Keith D. Noto, San Francisco, CA (US)

(21) Appl. No.: 17/825,220

(22) Filed: May 26, 2022

Related U.S. Application Data
(60) Provisional application No. 63/193,788, filed on May 27, 2021.

(51) Int. Cl. G06F 16/22; G16B 50/00

(52) U.S. Cl. CPC G06F 16/2237; G16B 50/00

(57) ABSTRACT

Disclosed herein relates to processes that identify segments of a target dataset that match segments of other datasets in a database. A computing server may encode the target dataset to generate a pair of encoded target bitmap sequences based on an encoding scheme. The encoding scheme defines encoding values based on homogeneity between the pair of data value sequences. The computing server may compare the pair of encoded target bitmap sequences with other pairs of encoded bitmap sequences to identify homogeneous mismatched locations. A homogeneous mismatched location may be a location where the target dataset and the other dataset in comparison are both homogeneous but have different types of homogeneity at the location. The computing server may identify a matched segment between the target dataset and one of the other datasets based on the homogeneous mismatched locations identified. The matched segment is contained within two homogeneous mismatched locations.

### CROSS-REFERENCE TO RELATED APPLICATION

The present application claims the benefit of U.S. Provisional Patent Application No. 63/193,788, filed on May 27, 2021, which is hereby incorporated by reference in its entirety.

### FIELD

The disclosed embodiments relate to identifying matched segments in datasets of a large-scale database and, more specifically, to an encoding scheme that speeds up the identification of matched segments.

### BACKGROUND

A large-scale database such as user profile and genetic database can include billions of data records. This type of database may allow users to build family trees, research their family history, and make meaningful discoveries about the lives of their ancestors. Users may try to identify relatives with datasets in the database. However, identifying relatives in the sheer amount of data is not a trivial task. Datasets associated with different individuals may not be connected without a proper determination of how the datasets are related. Comparing a large number of datasets without a concrete strategy may also be computationally infeasible because each dataset may also include a large number of data bits. Given an individual dataset and a database with datasets that are potentially related to the individual dataset, it is often challenging to identify a dataset in the database that is associated with the individual dataset.

In some cases, the computing server for the large-scale database may run algorithms in an attempt to identify the similarity of the genetic data among millions of users in the database. However, the genetic data for even a single individual can be quite large. Matching segments that are almost always not a perfect match adds complexity to the process. Running an effective algorithm that can quickly identify matches among individual datasets in a large-scale database can be challenging in many aspects including accuracy, speed, scalability, and memory usage.

### SUMMARY

Disclosed herein relates to example embodiments that identify one or more segments of a target dataset that match segments of other datasets in a database. In some embodiments, a computer-implemented method may include encoding the target dataset to generate a pair of encoded target bitmap sequences based on an encoding scheme. The target dataset includes a pair of data value sequences. The encoding scheme defines encoding values based on homogeneity between the pair of data value sequences. The pair of encoded target bitmap sequences includes a first encoded target bitmap sequence that encodes a first type of homogeneous locations and a second encoded target bitmap sequence that encodes a second type of homogeneous locations. The method may further include comparing the pair of encoded target bitmap sequences with other pairs of encoded bitmap sequences to identify homogeneous mismatched locations. The other encoded bitmap sequences may be generated from the other datasets in the database using the encoding scheme. A homogeneous mismatched location may be a location where the target dataset and the other dataset in comparison are both homogeneous but have different types of homogeneity at the location. The method may further include identifying a matched segment between the target dataset and one of the other datasets based on the homogeneous mismatched locations identified. The matched segment is contained within two homogeneous mismatched locations.

In yet another embodiment, a non-transitory computer-readable medium that is configured to store instructions is described. The instructions, when executed by one or more processors, cause the one or more processors to perform a process that includes steps described in the above computer-implemented methods or described in any embodiments of this disclosure. In yet another embodiment, a system may include one or more processors and a storage medium that is configured to store instructions. The instructions, when executed by one or more processors, cause the one or more processors to perform a process that includes steps described in the above computer-implemented methods or described in any embodiments of this disclosure.

### BRIEF DESCRIPTION OF THE DRAWINGS

FIG. 1 illustrates a diagram of a system environment of an example computing system, in accordance with an embodiment.

FIG. 2 is a block diagram of an architecture of an example computing system, in accordance with an embodiment.

FIG. 3 is a flowchart depicting an example process for identifying one or more segments of a target dataset that match segments of other datasets in a database, in accordance with some embodiments.

FIG. 4 is a conceptual diagram illustrating an example encoding scheme that may be used to encode a genetic dataset to generate a pair of encoded bitmap sequences and, in accordance with some embodiments.

FIG. 5 is a conceptual diagram illustrating the detection of an IBD segment shared between two individuals.

FIG. 6 is a flowchart illustrating an example fast IBD identification process 600 using a pre-scan step that compares bitmap sequences to eliminate mismatches, in accordance with some embodiments.

FIG. 7 is a conceptual diagram illustrating an example sampling process for turning a pair of bitmap sequences into a pair of sparse bitmap sequences, in accordance with some embodiments.

FIG. 8 is a conceptual diagram illustrating an example way of sampling such that each window in the sparse bitmap sequences similarly represents an approximately fixed length in the bitmap sequences.

FIG. 9A is a conceptual diagram illustrating an example process for comparing sparse bitmap sequences of two individuals to identify the homozygous mismatched locations of the two individuals, in accordance with some embodiments.

FIG. 9B is a conceptual diagram illustrating a comparison process for comparing two bitmap sequences to identify homozygous mismatch, in accordance with some embodiments.

FIG. 10 is a flowchart depicting an example process for determining a matched segment, in accordance with some embodiments.

FIG. 11 is a block diagram of an example computing device, in accordance with an embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The figures (FIGs.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

In some embodiment, an encoding scheme that increases the speed of identifying matched segments in datasets is described. The encoding scheme may turn a dataset to a pair of homozygous bitmap sequences. The pair of bitmap sequences each encode the homozygous locations of different type of data values. For example, the first bitmap sequence of the pair carries information on the locations of a first type of data values (e.g., major alleles) of an individual's dataset. The second bitmap sequence of the pair carries information on the locations of a second type of data values (e.g., minor alleles) of the individual's dataset. The encoding homozygous bitmap sequences may be used to quickly identify matched segments of two individuals' datasets such as using pairwise AND operations.

### Example System Environment

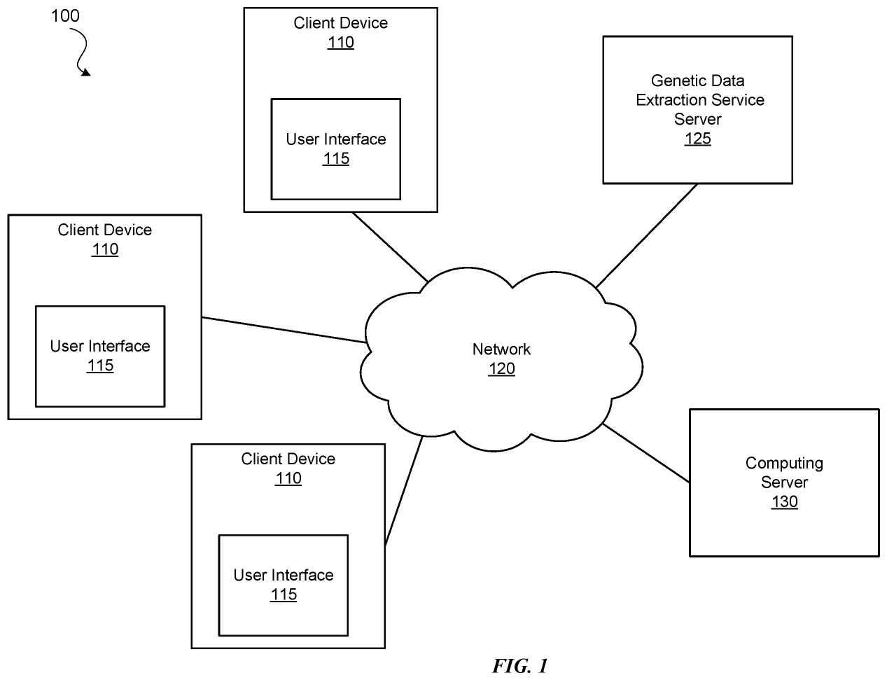

FIG. 1 illustrates a diagram of a system environment 100 of an example computing server 130, in accordance with an embodiment. The system environment 100 shown in FIG. 1 includes one or more client devices 110, a network 120, a genetic data extraction service server 125, and a computing server 130. In various embodiments, the system environment 100 may include fewer or additional components. The system environment 100 may also include different components.

The client devices 110 are one or more computing devices capable of receiving user input as well as transmitting and/or receiving data via a network 120. Example computing devices include desktop computers, laptop computers, personal digital assistants (PDAs), smartphones, tablets, wearable electronic devices (e.g., smartwatches), smart household appliance (e.g., smart televisions, smart speakers, smart home hubs), Internet of Things (IoT) devices or other suitable electronic devices. A client device 110 communicates to other components via the network 120. Users may be customers of the computing server 130 or any individuals who access the system of the computing server 130, such as an online website or a mobile application. In one embodiment, a client device 110 executes an application that launches a graphical user interface (GUI) for a user of the client device 110 to interact with the computing server 130. The GUI may be an example of a user interface 115. A client device 110 may also execute a web browser application to enable interactions between the client device 110 and the computing server 130 via the network 120. In another embodiment, the user interface 115 may take the form of a software application published by the computing server 130 and installed on the user device 110. In yet another embodiment, a client device 110 interacts with the computing server 130 through an application programming interface (API) running on a native operating system of the client device 110, such as IOS or ANDROID.

The network 120 provides connections to the components of the system environment 100 through one or more sub-networks, which may include any combination of local area and/or wide area networks, using both wired and/or wireless communication systems. In one embodiment, a network 120 uses standard communications technologies and/or protocols. For example, a network 120 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 120 may be represented using any suitable format, such as hypertext markup language (HTML) or extensible markup language (XML). In some embodiments, all or some of the communication links of a network 120 may be encrypted using any suitable technique or techniques such as secure sockets layer (SSL), transport layer security (TLS), virtual private networks (VPNs), Internet Protocol security (IPsec), etc. The network 120 also includes links and packet switching networks such as the Internet.

Individuals, who may be customers of a company operating the computing server 130, provide biological samples for analysis of their genetic data. Individuals may also be referred to as users. In one embodiment, an individual uses a sample collection kit to provide a biological sample (e.g., saliva, blood, hair, tissue) from which genetic data is extracted and determined according to nucleotide processing techniques such as amplification and sequencing. Amplification may include using polymerase chain reaction (PCR) to amplify segments of nucleotide samples. Sequencing may include sequencing of deoxyribonucleic acid (DNA) sequencing, ribonucleic acid (RNA) sequencing, etc. Suitable sequencing techniques may include Sanger sequencing and massively parallel sequencing such as various next-generation sequencing (NGS) techniques including whole genome sequencing, pyrosequencing, sequencing by synthesis, sequencing by ligation, and ion semiconductor sequencing. In one embodiment, a set of SNPs (e.g., 300,000) that are shared between different array platforms (e.g., Illumina OmniExpress Platform and Illumina HumanHap 650Y Platform) may be obtained as the genetic data. Genetic data extraction service server 125 receives biological samples from users of the computing server 130. The genetic data extraction service server 125 performs sequencing of the biological samples and determines the base pair sequences of the individuals. The genetic data extraction service server 125 generates the genetic data of the individuals based on the sequencing results. The genetic data may include data sequenced from DNA or RNA and may include base pairs from coding and/or noncoding regions of DNA.

The genetic data may take different forms and include information regarding various biomarkers of an individual. For example, in one embodiment, the genetic data may be the base pair sequence of an individual. The base pair sequence may include the whole genome or a part of the genome such as certain genetic loci of interest. In another embodiment, the genetic data extraction service server 125 may determine genotypes from sequencing results, for example by identifying genotype values of single nucleotide polymorphisms (SNPs) present within the DNA. The results in this example may include a sequence of genotypes corresponding to various SNP sites. A SNP site may also be referred to as a SNP loci. A genetic locus is a segment of a genetic sequence. A locus can be a single site or a longer stretch. The segment can be a single base long or multiple bases long. In one embodiment, the genetic data extraction service server 125 may perform data pre-processing of the genetic data to convert raw sequences of base pairs to sequences of genotypes at target SNP sites. Since a typical human genome may differ from a reference human genome at only several million SNP sites (as opposed to billions of base pairs in the whole genome), the genetic data extraction service server 125 may extract only the genotypes at a set of target SNP sites and transmit the extracted data to the computing server 130 as the genetic dataset of an individual. SNPs, base pair sequence, genotype, haplotype, RNA sequences, protein sequences, phenotypes are examples of biomarkers.

The computing server 130 performs various analyses of the genetic data, genealogical data, and users' survey responses to generate results regarding the phenotypes and genealogy of users of computing server 130. Depending on the embodiments, the computing server 130 may also be referring to as an online server, a personal genetic service server, a genealogy server, a family tree building server, and/or a social networking system. The computing server 130 receives genetic data from the genetic data extraction service server 125 and stores the genetic data in the data store of the computing server 130. The computing server 130 may analyze the data to generate results regarding the genetics or genealogy of users. The results regarding the genetics or genealogy of users may include the ethnicity compositions of users, paternal and maternal genetic analysis, identification or suggestion of potential family relatives, ancestor information, analyses of DNA data, potential or identified traits such as phenotypes of users (e.g., diseases, appearance traits, other genetic characteristics, and other non-genetic characteristics including social characteristics), etc. The computing server 130 may present or cause the user interface 115 to present the results to the users through a GUI displayed at the client device 110. The results may include graphical elements, textual information, data, charts, and other elements such as family trees.

In one embodiment, the computing server 130 also allows various users to create one or more genealogical profiles of the user. The genealogical profile may include a list of individuals (e.g., ancestors, relatives, friends, and other people of interest) who are added or selected by the user or suggested by the computing server 130 based on the genealogical records and/or genetic records. The user interface 115 controlled by or in communication with the computing server 130 may display the individuals in a list or as a family tree such as in the form of a pedigree chart. In one embodiment, subject to user's privacy setting and authorization, the computing server 130 may allow information generated from the user's genetic dataset to be linked to the user profile and to one or more of the family trees. The users may also authorize the computing server 130 to analyze their genetic dataset and allow their profiles to be discovered by other users.

### Example Computing Server Architecture

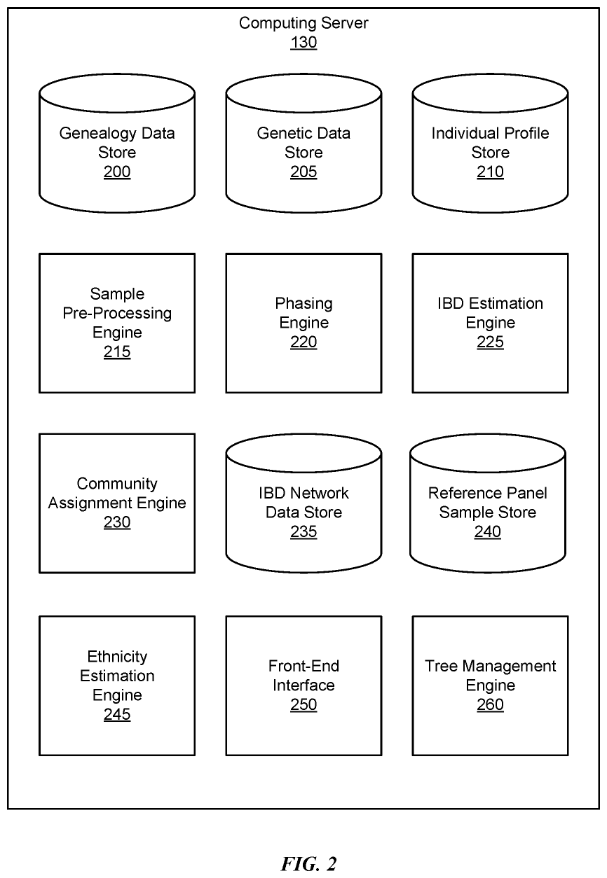

FIG. 2 is a block diagram of an architecture of an example computing server 130, in accordance with an embodiment. In the embodiment shown in FIG. 2, the computing server 130 includes a genealogy data store 200, a genetic data store 205, an individual profile store 210, a sample pre-processing engine 215, a phasing engine 220, an identity by descent (IBD) estimation engine 225, a community assignment engine 230, an IBD network data store 235, a reference panel sample store 240, an ethnicity estimation engine 245, and a front-end interface 250. The functions of the computing server 130 may be distributed among the elements in a different manner than described. In various embodiments, the computing server 130 may include different components and fewer or additional components. Each of the various data stores may be a single storage device, a server controlling multiple storage devices, or a distributed network that is accessible through multiple nodes (e.g., a cloud storage system).

The computing server 130 stores various data of different individuals, including genetic data, genealogical data, and survey response data. The computing server 130 processes the genetic data of users to identify shared identity-by-descent (IBD) segments between individuals. The genealogical data and survey response data may be part of user profile data. The amount and type of user profile data stored for each user may vary based on the information of a user, which is provided by the user as she creates an account and profile at a system operated by the computing server 130 and continues to build her profile, family tree, and social network at the system and to link her profile with her genetic data. Users may provide data via the user interface 115 of a client device 110. Initially and as a user continues to build her genealogical profile, the user may be prompted to answer questions related to the basic information of the user (e.g., name, date of birth, birthplace, etc.) and later on more advanced questions that may be useful for obtaining additional genealogical data. The computing server 130 may also include survey questions regarding various traits of the users such as the users' phenotypes, characteristics, preferences, habits, lifestyle, environment, etc.

Genealogical data may be stored in the genealogical data store 200 and may include various types of data that are related to tracing family relatives of users. Examples of genealogical data include names (first, last, middle, suffixes), gender, birth locations, date of birth, date of death, marriage information, spouse's information kinships, family history, dates and places for life events (e.g., birth and death), other vital data, and the like. In some instances, family history can take the form of a pedigree of an individual (e.g., the recorded relationships in the family). The family tree information associated with an individual may include one or more specified nodes. Each node in the family tree represents the individual, an ancestor of the individual who might have passed down genetic material to the individual, and the individual's other relatives including siblings, cousins, offspring in some cases. Genealogical data may also include connections and relationships among users of the computing server 130. The information related to the connections among a user and her relatives that may be associated with a family tree may also be referred to as pedigree data or family tree data.

In addition to user-input data, genealogical data may also take other forms that are obtained from various sources such as public records and third-party data collectors. For example, genealogical records from public sources include birth records, marriage records, death records, census records, court records, probate records, adoption records, obituary records, etc. Likewise, genealogical data may include data from one or more of a pedigree of an individual, the Ancestry World Tree system, a Social Security Death Index database, the World Family Tree system, a birth certificate database, a death certificate database, a marriage certificate database, an adoption database, a draft registration database, a veterans database, a military database, a property records database, a census database, a voter registration database, a phone database, an address database, a newspaper database, an immigration database, a family history records database, a local history records database, a business registration database, a motor vehicle database, and the like.

Furthermore, the genealogical data store 200 may also include relationship information inferred from the genetic data stored in the genetic data store 205 and information received from the individuals. For example, the relationship information may indicate which individuals are genetically related, how they are related, how many generations back they share common ancestors, lengths and locations of IBD segments shared, which genetic communities an individual is a part of, variants carried by the individual, and the like.

The computing server 130 maintains genetic datasets of individuals in the genetic data store 205. A genetic dataset of an individual may be a digital dataset of nucleotide data (e.g., SNP data) and corresponding metadata. A genetic dataset may contain data of the whole or portions of an individual's genome. The genetic data store 205 may store a pointer to a location associated with the genealogical data store 200 associated with the individual. A genetic dataset may take different forms. In one embodiment, a genetic dataset may take the form of a base pair sequence of the sequencing result of an individual. A base pair sequence dataset may include the whole genome of the individual (e.g., obtained from a whole-genome sequencing) or some parts of the genome (e.g., genetic loci of interest).

In another embodiment, a genetic dataset may take the form of sequences of genetic markers. Examples of genetic markers may include target SNP loci (e.g., allele sites) filtered from the sequencing results. A SNP locus that is single base pair long may also be referred to a SNP site. A SNP locus may be associated with a unique identifier. The genetic dataset may be in a form of diploid data that includes a sequencing of genotypes, such as genotypes at the target SNP loci, or the whole base pair sequence that includes genotypes at known SNP loci and other base pair sites that are not commonly associated with known SNPs. The diploid dataset may be referred to as a genotype dataset or a genotype sequence. Genotype may have a different meaning in various contexts. In one context, an individual's genotype may refer to a collection of diploid alleles of an individual. In other contexts, a genotype may be a pair of alleles present on two chromosomes for an individual at a given genetic marker such as a SNP site.

Genotype data for a SNP site may include a pair of alleles. The pair of alleles may be homozygous (e.g., A-A or G-G) or heterozygous (e.g., A-T, C-T). Instead of storing the actual nucleotides, the genetic data store 205 may store genetic data that are converted to bits. For a given SNP site, oftentimes only two nucleotide alleles (instead of all 4) are observed. As such, a 2-bit number may represent a SNP site. For example, 00 may represent homozygous first alleles, 11 may represent homozygous second alleles, and 01 or 10 may represent heterozygous alleles. A separate library may store what nucleotide corresponds to the first allele and what nucleotide corresponds to the second allele at a given SNP site.

A diploid dataset may also be phased into two sets of haploid data, one corresponding to a first parent side and another corresponding to a second parent side. The phased datasets may be referred to as haplotype datasets or haplotype sequences. Similar to genotype, haplotype may have a different meaning in various contexts. In one context, a haplotype may also refer to a collection of alleles that corresponds to a genetic segment. In other contexts, a haplotype may refer to a specific allele at a SNP site. For example, a sequence of haplotypes may refer to a sequence of alleles of an individual that are inherited from a parent.

The individual profile store 210 stores profiles and related metadata associated with various individuals appeared in the computing server 130. A computing server 130 may use unique individual identifiers to identify various users and other non-users that might appear in other data sources such as ancestors or historical persons who appear in any family tree or genealogical database. A unique individual identifier may a hash of certain identification information of an individual, such as a user's account name, user's name, date of birth, location of birth, or any suitable combination of the information. The profile data related to an individual may be stored as metadata associated with an individual's profile. For example, the unique individual identifier and the metadata may be stored as a key-value pair using the unique individual identifier as a key.

An individual's profile data may include various kinds of information related to the individual. The metadata about the individual may include one or more pointer associating genetic datasets such as genotype and phased haplotype data of the individual that are saved in the genetic data store 205. The metadata about the individual may also individual information related to family trees and pedigree datasets that include the individual. The profile data may further include declarative information about the user that was authorized by the user to be shared and may also include information inferred by the computing server 130. Other examples of information stored in a user profile may include biographic, demographic, and other types of descriptive information such as work experience, educational history, gender, hobbies, or preferences, location and the like. In one embodiment, the user profile data may also include one or more photos of the users and photos of relatives (e.g., ancestors) of the users that are uploaded by the users. A user may authorize the computing server 130 to analyze one or more photos to extract information, such as the user's or relative's appearance traits (e.g., blue eyes, curved hair, etc.), from the photos. The appearance traits and other information extracted from the photos may also be saved in the profile store. In some cases, the computing server may allow users to upload many different photos of the users, their relatives, and even friends. User profile data may also be obtained from other suitable sources, including historical records (e.g., records related to an ancestor), medical records, military records, photographs, other records indicating one or more traits, and other suitable recorded data.

For example, the computing server 130 may present various survey questions to its users from time to time. The responses to the survey questions may be stored at individual profile store 210. The survey questions may be related to various aspects of the users and the users' families. Some survey questions may be related to users' phenotypes, while other questions may be related to environmental factors of the users.

Survey questions may concern health or disease-related phenotypes, such as questions related to the presence or absence of genetic diseases or disorders, inheritable diseases or disorders, or other common diseases or disorders that have a family history as one of the risk factors, questions regarding any diagnosis of increased risk of any diseases or disorders, and questions concerning wellness-related issues such as a family history of obesity, family history of causes of death, etc. The diseases identified by the survey questions may be related to single-gene diseases or disorders that are caused by a single-nucleotide variant, an insertion, or a deletion. The diseases identified by the survey questions may also be multifactorial inheritance disorders that may be caused by a combination of environmental factors and genes. Examples of multifactorial inheritance disorders may include heart disease, Alzheimer's diseases, diabetes, cancer, and obesity. The computing server 130 may obtain data of a user's disease-related phenotypes from survey questions of the health history of the user and her family and also from health records uploaded by the user.

Survey questions also may be related to other types of phenotypes such as appearance traits of the users. A survey regarding appearance traits and characteristics may include questions related to eye color, iris pattern, freckles, chin types, finger length, dimple chin, earlobe types, hair color, hair curl, skin pigmentation, susceptibility to skin burn, bitter taste, male baldness, baldness pattern, presence of unibrow, presence of wisdom teeth, height, and weight. A survey regarding other traits also may include questions related to users' taste and smell such as the ability to taste bitterness, asparagus smell, cilantro aversion, etc. A survey regarding traits may further include questions related to users' body conditions such as lactose tolerance, caffeine consumption, malaria resistance, norovirus resistance, muscle performance, alcohol flush, etc. Other survey questions regarding a person's physiological or psychological traits may include vitamin traits and sensory traits such as the ability to sense an asparagus metabolite. Traits may also be collected from historical records, electronic health records and electronic medical records.

The computing server 130 also may present various survey questions related to the environmental factors of users. In this context, an environmental factor may be a factor that is not directly connected to the genetics of the users. Environmental factors may include users' preferences, habits, and lifestyles. For example, a survey regarding users' preferences may include questions related to things and activities that users like or dislike, such as types of music a user enjoys, dancing preference, party-going preference, certain sports that a user plays, video games preferences, etc. Other questions may be related to the users' diet preference such as like or dislike a certain type of food (e.g., ice cream, egg). A survey related to habits and lifestyle may include questions regarding smoking habits, alcohol consumption and frequency, daily exercise duration, sleeping habits (e.g., morning person versus night person), sleeping cycles and problems, hobbies, and travel preferences. Additional environmental factors may include diet amount (calories, macronutrients), physical fitness abilities (e.g. stretching, flexibility, heart rate recovery), family type (adopted family or not, has siblings or not, lived with extended family during childhood), property and item ownership (has home or rents, has a smartphone or doesn't, has a car or doesn't).

Surveys also may be related to other environmental factors such as geographical, social-economic, or cultural factors. Geographical questions may include questions related to the birth location, family migration history, town, or city of users' current or past residence. Social-economic questions may be related to users' education level, income, occupations, self-identified demographic groups, etc. Questions related to culture may concern users' native language, language spoken at home, customs, dietary practices, etc. Other questions related to users' cultural and behavioral questions are also possible.

For any survey questions asked, the computing server 130 may also ask an individual the same or similar questions regarding the traits and environmental factors of the ancestors, family members, other relatives or friends of the individual. For example, a user may be asked about the native language of the user and the native languages of the user's parents and grandparents. A user may also be asked about the health history of his or her family members.

In addition to storing the survey data in the individual profile store 210, the computing server 130 may store some responses that correspond to data related to genealogical and genetics respectively to genealogical data store 200 and genetic data store 205.

The user profile data, photos of users, survey response data, the genetic data, and the genealogical data may subject to the privacy and authorization setting from the users to specify any data related to the users can be accessed, stored, obtained, or otherwise used. For example, when presented with a survey question, a user may select to answer or skip the question. The computing server 130 may present users from time to time information regarding users' selection of the extent of information and data shared. The computing server 130 also may maintain and enforce one or more privacy settings for users in connection with the access of the user profile data, photos, genetic data, and other sensitive data. For example, the user may pre-authorize the access of the data and may change the setting as wish. The privacy settings also may allow a user to specify (e.g., by opting out, by not opting in) whether the computing server 130 may receive, collect, log, or store particular data associated with the user for any purpose. A user may restrict her data at various levels. For example, on one level, the data may not be accessed by the computing server 130 for purposes other than displaying the data in the user's own profile. On another level, the user may authorize anonymization of her data and participate in studies and researches conducted by the computing server 130 such as a large-scale genetic study. On yet another level, the user may turn some portions of her genealogical data public to allow the user to be discovered by other users (e.g., potential relatives) and be connected in one or more family trees. Access or sharing of any information or data in the computing server 130 may also be subject to one or more similar privacy policies. A user's data and content objects in the computing server 130 may also be associated with different levels of restriction. The computing server 130 may also provide various notification features to inform and remind users of their privacy and access settings. For example, when privacy settings for a data entry allow a particular user or other entities to access the data, the data may be described as being “visible,” “public,” or other suitable labels, contrary to a “private” label.

In some cases, the computing server 130 may have a heightened privacy protection on certain types of data and data related to certain vulnerable groups. In some cases, the heightened privacy settings may strictly prohibit the use, analysis, sharing of data related to a certain vulnerable group. In other cases, the heightened privacy settings may specify that data subject to those settings require prior approval for access, publication, or other use. In some cases, the computing server 130 may provide the heightened privacy as a default setting for certain types of data, such as genetic data or any data that the user marks as sensitive. The user may opt in for sharing of those data or change the default privacy settings. In other cases, the heightened privacy settings may apply across the board for all data of certain groups of users. For example, if the computing server 130 determines that the user is a minor or has recognized that a picture of a minor is uploaded, the computing server 130 may designate all profile data associated with the minor as sensitive. In those cases, the computing server 130 may have one or more extra steps in seeking and confirming any sharing or use of the sensitive data.

The sample pre-processing engine 215 receives and pre-processes data received from various sources to change the data into a format used by the computing server 130. For genealogical data, the sample pre-processing engine 215 may receive data from an individual via the user interface 115 of the client device 110. To collect the user data (e.g., genealogical and survey data), the computing server 130 may cause an interactive user interface on the client device 110 to display interface elements in which users can provide genealogical data and survey data. Additional data may be obtained from scans of public records. The data may be manually provided or automatically extracted via, for example, optical character recognition (OCR) performed on census records, town or government records, or any other item of printed or online material. Some records may be obtained by digitalizing written records such as older census records, birth certificates, death certificates, etc.

The sample pre-processing engine 215 may also receive raw data from genetic data extraction service server 125. The genetic data extraction service server 125 may perform laboratory analysis of biological samples of users and generate sequencing results in the form of digital data. The sample pre-processing engine 215 may receive the raw genetic datasets from the genetic data extraction service server 125. The human genome mutation rate is estimated to be 1.1*10\hat{ }−8 per site per generation. This may lead to a variant of approximately every 300 base pairs. Most of the mutations that are passed down to descendants are related to single-nucleotide polymorphism (SNP). SNP is a substitution of a single nucleotide that occurs at a specific position in the genome. The sample pre-processing engine 215 may convert the raw base pair sequence into a sequence of genotypes of target SNP sites. Alternatively, the pre-processing of this conversion may be performed by the genetic data extraction service server 125. The sample pre-processing engine 215 identifies autosomal SNPs in an individual's genetic dataset. In one embodiment, the SNPs may be autosomal SNPs. In one embodiment, 700,000 SNPs may be identified in an individual's data and may be stored in genetic data store 205. Alternatively, in one embodiment, a genetic dataset may include at least 10,000 SNP sites. In another embodiment, a genetic dataset may include at least 100,000 SNP sites. In yet another embodiment, a genetic dataset may include at least 300,000 SNP sites. In yet another embodiment, a genetic dataset may include at least 1,000,000 SNP sites. The sample pre-processing engine 215 may also convert the nucleotides into bits. The identified SNPs, in bits or in other suitable formats, may be provided to the phasing engine 220 which phases the individual's diploid genotypes to generate a pair of haplotypes for each user.

The phasing engine 220 phases diploid genetic dataset into a pair of haploid genetic datasets and may perform imputation of SNP values at certain sites whose alleles are missing. An individual's haplotype may refer to a collection of alleles (e.g., a sequence of alleles) that are inherited from a parent.

Phasing may include a process of determining the assignment of alleles (particularly heterozygous alleles) to chromosomes. Owing to sequencing conditions and other constraints, a sequencing result often includes data regarding a pair of alleles at a given SNP locus of a pair of chromosomes but may not be able to distinguish which allele belongs to which specific chromosome. The phasing engine 220 uses a genotype phasing algorithm to assign one allele to a first chromosome and another allele to another chromosome. The genotype phasing algorithm may be developed based on an assumption of linkage disequilibrium (LD), which states that haplotype in the form of a sequence of alleles tends to cluster together. The phasing engine 220 is configured to generate phased sequences that are also commonly observed in many other samples. Put differently, haplotype sequences of different individuals tend to cluster together. A haplotype-cluster model may be generated to determine the probability distribution of a haplotype that includes a sequence of alleles. The haplotype-cluster model may be trained based on labeled data that includes known phased haplotypes from a trio (parents and a child). A trio is used as a training sample because the correct phasing of the child is almost certain by comparing the child's genotypes to the parent's genetic datasets. The haplotype-cluster model may be generated iteratively along with the phasing process with a large number of unphased genotype datasets. The haplotype-cluster model may also be used to impute one or more missing data.

By way of example, the phasing engine 220 may use a directed acyclic graph model such as a hidden Markov model (HMM) to perform phasing of a target genotype dataset. The directed acyclic graph may include multiple levels, each level having multiple nodes representing different possibilities of haplotype clusters. An emission probability of a node, which may represent the probability of having a particular haplotype cluster given an observation of the genotypes may be determined based on the probability distribution of the haplotype-cluster model. A transition probability from one node to another may be initially assigned to a non-zero value and be adjusted as the directed acyclic graph model and the haplotype-cluster model are trained. Various paths are possible in traversing different levels of the directed acyclic graph model. The phasing engine 220 determines a statistically likely path, such as the most probable path or a probable path that is at least more likely than 95% of other possible paths, based on the transition probabilities and the emission probabilities. A suitable dynamic programming algorithm such as the Viterbi algorithm may be used to determine the path. The determined path may represent the phasing result. U.S. Pat. No. 10,679,729, entitled “Haplotype Phasing Models,” granted on Jun. 9, 2020, describes example embodiments of haplotype phasing.

The IBD estimation engine 225 estimates the amount of shared genetic segments between a pair of individuals based on phased genotype data (e.g., haplotype datasets) that are stored in the genetic data store 205. IBD segments may be segments identified in a pair of individuals that are putatively determined to be inherited from a common ancestor. The IBD estimation engine 225 retrieves a pair of haplotype datasets for each individual. The IBD estimation engine 225 may divide each haplotype dataset sequence into a plurality of windows. Each window may include a fixed number of SNP sites (e.g., about 100 SNP sites). The IBD estimation engine 225 identifies one or more seed windows in which the alleles at all SNP sites in at least one of the phased haplotypes between two individuals are identical. The IBD estimation engine 225 may expand the match from the seed windows to nearby windows until the matched windows reach the end of a chromosome or until a homozygous mismatch is found, which indicates the mismatch is not attributable to potential errors in phasing or imputation. The IBD estimation engine 225 determines the total length of matched segments, which may also be referred to as IBD segments. The length may be measured in the genetic distance in the unit of centimorgans (cM). A unit of centimorgan may be a genetic length. For example, two genomic positions that are one cM apart may have a 1% chance during each meiosis of experiencing a recombination event between the two positions. The computing server 130 may save data regarding individual pairs who share a length of IBD segments exceeding a predetermined threshold (e.g., 6 cM), in a suitable data store such as in the genealogical data store 200. U.S. Pat. No. 10,114,922, entitled “Identifying Ancestral Relationships Using a Continuous stream of Input,” granted on Oct. 30, 2018, and U.S. Pat. No. 10,720,229, entitled “Reducing Error in Predicted Genetic Relationships,” granted on Jul. 21, 2020, describe example embodiments of IBD estimation.

Typically, individuals who are closely related share a relatively large number of IBD segments, and the IBD segments tend to have longer lengths (individually or in aggregate across one or more chromosomes). In contrast, individuals who are more distantly related share relatively fewer IBD segments, and these segments tend to be shorter (individually or in aggregate across one or more chromosomes). For example, while close family members often share upwards of 71 cM of IBD (e.g., third cousins), more distantly related individuals may share less than 12 cM of IBD. The extent of relatedness in terms of IBD segments between two individuals may be referred to as IBD affinity. For example, the IBD affinity may be measured in terms of the length of IBD segments shared between two individuals.

Community assignment engine 230 assigns individuals to one or more genetic communities based on the genetic data of the individuals. A genetic community may correspond to an ethnic origin or a group of people descended from a common ancestor. The granularity of genetic community classification may vary depending on embodiments and methods used to assign communities. For example, in one embodiment, the communities may be African, Asian, European, etc. In another embodiment, the European community may be divided into Irish, German, Swedes, etc. In yet another embodiment, the Irish may be further divided into Irish in Ireland, Irish immigrated to America in 1800, Irish immigrated to America in 1900, etc. The community classification may also depend on whether a population is admixed or unadmixed. For an admixed population, the classification may further be divided based on different ethnic origins in a geographical region.

Community assignment engine 230 may assign individuals to one or more genetic communities based on their genetic datasets using machine learning models trained by unsupervised learning or supervised learning. In an unsupervised approach, the community assignment engine 230 may generate data representing a partially connected undirected graph. In this approach, the community assignment engine 230 represents individuals as nodes. Some nodes are connected by edges whose weights are based on IBD affinity between two individuals represented by the nodes. For example, if the total length of two individuals' shared IBD segments does not exceed a predetermined threshold, the nodes are not connected. The edges connecting two nodes are associated with weights that are measured based on the IBD affinities. The undirected graph may be referred to as an IBD network. The community assignment engine 230 uses clustering techniques such as modularity measurement (e.g., the Louvain method) to classify nodes into different clusters in the IBD network. Each cluster may represent a community. The community assignment engine 230 may also determine sub-clusters, which represent sub-communities. The computing server 130 saves the data representing the IBD network and clusters in the IBD network data store 235. U.S. Pat. No. 10,223,498, entitled “Discovering Population Structure from Patterns of Identity-By-Descent,” granted on Mar. 5, 2019, describes example embodiments of community detection and assignment.

The community assignment engine 230 may also assign communities using supervised techniques. For example, genetic datasets of known genetic communities (e.g., individuals with confirmed ethnic origins) may be used as training sets that have labels of the genetic communities. Supervised machine learning classifiers, such as logistic regressions, support vector machines, random forest classifiers, and neural networks may be trained using the training set with labels. A trained classifier may distinguish binary or multiple classes. For example, a binary classifier may be trained for each community of interest to determine whether a target individual's genetic dataset belongs or does not belong to the community of interest. A multi-class classifier such as a neural network may also be trained to determine whether the target individual's genetic dataset most likely belongs to one of several possible genetic communities.

Reference panel sample store 240 stores reference panel samples for different genetic communities. A reference panel sample is a genetic data of an individual whose genetic data is the most representative of a genetic community. The genetic data of individuals with the typical alleles of a genetic community may serve as reference panel samples. For example, some alleles of genes may be over-represented (e.g., being highly common) in a genetic community. Some genetic datasets include alleles that are commonly present among members of the community. Reference panel samples may be used to train various machine learning models in classifying whether a target genetic dataset belongs to a community, in determining the ethnic composition of an individual, and in determining the accuracy in any genetic data analysis, such as by computing a posterior probability of a classification result from a classifier.

A reference panel sample may be identified in different ways. In one embodiment, an unsupervised approach in community detection may apply the clustering algorithm recursively for each identified cluster until the sub-clusters contain a number of nodes that are smaller than a threshold (e.g., contains fewer than 1000 nodes). For example, the community assignment engine 230 may construct a full IBD network that includes a set of individuals represented by nodes and generate communities using clustering techniques. The community assignment engine 230 may randomly sample a subset of nodes to generate a sampled IBD network. The community assignment engine 230 may recursively apply clustering techniques to generate communities in the sampled IBD network. The sampling and clustering may be repeated for different randomly generated sampled IBD networks for various runs. Nodes that are consistently assigned to the same genetic community when sampled in various runs may be classified as a reference panel sample. The community assignment engine 230 may measure the consistency in terms of a predetermined threshold. For example, if a node is classified to the same community 95% (or another suitable threshold) of times whenever the node is sampled, the genetic dataset corresponding to the individual represented by the node may be regarded as a reference panel sample. Additionally, or alternatively, the community assignment engine 230 may select N most consistently assigned nodes as a reference panel for the community.

Other ways to generate reference panel samples are also possible. For example, the computing server 130 may collect a set of samples and gradually filter and refine the samples until high-quality reference panel samples are selected. For example, a candidate reference panel sample may be selected from an individual whose recent ancestors are born at a certain birthplace. The computing server 130 may also draw sequence data from the Human Genome Diversity Project (HGDP). Various candidates may be manually screened based on their family trees, relatives' birth location, other quality control. Principal component analysis may be used to creates clusters of genetic data of the candidates. Each cluster may represent an ethnicity. The predictions of the ethnicity of those candidates may be compared to the ethnicity information provided by the candidates to perform further screening.

The ethnicity estimation engine 245 estimates the ethnicity composition of a genetic dataset of a target individual. The genetic datasets used by the ethnicity estimation engine 245 may be genotype datasets or haplotype datasets. For example, the ethnicity estimation engine 245 estimates the ancestral origins (e.g., ethnicity) based on the individual's genotypes or haplotypes at the SNP sites. To take a simple example of three ancestral populations corresponding to African, European and Native American, an admixed user may have nonzero estimated ethnicity proportions for all three ancestral populations, with an estimate such as [0.05, 0.65, 0.30], indicating that the user's genome is 5% attributable to African ancestry, 65% attributable to European ancestry and 30% attributable to Native American ancestry. The ethnicity estimation engine 245 generates the ethnic composition estimate and stores the estimated ethnicities in a data store of computing server 130 with a pointer in association with a particular user.

In one embodiment, the ethnicity estimation engine 245 divides a target genetic dataset into a plurality of windows (e.g., about 1000 windows). Each window includes a small number of SNPs (e.g., 300 SNPs). The ethnicity estimation engine 245 may use a directed acyclic graph model to determine the ethnic composition of the target genetic dataset. The directed acyclic graph may represent a trellis of an inter-window hidden Markov model (HMM). The graph includes a sequence of a plurality of node groups. Each node group, representing a window, includes a plurality of nodes. The nodes representing different possibilities of labels of genetic communities (e.g., ethnicities) for the window. A node may be labeled with one or more ethnic labels. For example, a level includes a first node with a first label representing the likelihood that the window of SNP sites belongs to a first ethnicity and a second node with a second label representing the likelihood that the window of SNPs belongs to a second ethnicity. Each level includes multiple nodes so that there are many possible paths to traverses the directed acyclic graph.

The nodes and edges in the directed acyclic graph may be associated with different emission probabilities and transition probabilities. An emission probability associated with a node represents the likelihood that the window belongs to the ethnicity labeling the node given the observation of SNPs in the window. The ethnicity estimation engine 245 determines the emission probabilities by comparing SNPs in the window corresponding to the target genetic dataset to corresponding SNPs in the windows in various reference panel samples of different genetic communities stored in the reference panel sample store 240. The transition probability between two nodes represents the likelihood of transition from one node to another across two levels. The ethnicity estimation engine 245 determines a statistically likely path, such as the most probable path or a probable path that is at least more likely than 95% of other possible paths, based on the transition probabilities and the emission probabilities. A suitable dynamic programming algorithm such as the Viterbi algorithm or the forward-backward algorithm may be used to determine the path. After the path is determined, the ethnicity estimation engine 245 determines the ethnic composition of the target genetic dataset by determining the label compositions of the nodes that are included in the determined path. U.S. Pat. No. 10,558,930, entitled “Local Genetic Ethnicity Determination System,” granted on Feb. 11, 2020, describes example embodiments of ethnicity estimation.

The front-end interface 250 displays various results determined by the computing server 130. The results and data may include the IBD affinity between a user and another individual, the community assignment of the user, the ethnicity estimation of the user, phenotype prediction and evaluation, genealogical data search, family tree and pedigree, relative profile and other information. The front-end interface 250 may allow users to manage their profile and data trees (e.g., family trees). The users may view various public family trees in stored in the computing server 130 and search for individuals and their genealogical data via the front-end interface 250. The computing server 130 may suggest or allow the user to manually review and select potential related individuals (e.g., relatives, ancestors, close family members) to add to the user's data tree. The front-end interface 250 may be a graphical user interface (GUI) that displays various information and graphical elements. The front-end interface 250 may take different forms. In one case, the front-end interface 250 may be a software application that can be displayed on an electronic device such as a computer or a smartphone. The software application may be developed by the entity controlling the computing server 130 and be downloaded and installed at the client device 110. In another case, the front-end interface 250 may take the form of a webpage interface of the computing server 130 that allows users to access their family tree and genetic analysis results through web browsers. In yet another case, the front-end interface 250 may provide an application program interface (API).

The tree management engine 260 performs computations and other processes related to users' management of their data trees such as family trees. The tree management engine 260 may allow a user to build a data tree from scratch or to link the user to existing data trees. In some embodiments, the tree management engine 260 may suggest a connection between a target individual and a family tree that exists in the family tree database by identifying potential family trees for the target individual and identifying one or more most probable positions in a potential family tree. A user (target individual) may wish to identify family trees to which he or she may potentially belong. Linking a user to a family tree or building a family may be performed automatically, manually, or using techniques with a combination of both. In an embodiment of an automatic tree matching, the tree management engine 260 may receive a genetic dataset from the target individual as input and search related individuals that are IBD-related to the target individual. The tree management engine 260 may identify common ancestors. Each common ancestor may be a common to the target individual and one of the related individuals. The tree management engine 260 may in turn output potential family trees to which the target individual may belong by retrieving family trees that include a common ancestor and an individual who is IBD-related to the target individual. The tree management engine 260 may further identify one or more probable positions in one of the potential family trees based on information associated with matched genetic data between the target individual and DNA test takers in the potential family trees through one or more machine learning models or other heuristic algorithms. For example, the tree management engine 260 may try putting the target individual in various possible location in the family tree and determine the highest probability position(s) based on the genetic datasets of the target individual and other DNA test takes in the family tree and based on genealogical data available to the tree management engine 260. The tree management engine 260 may provide one or more family trees from which the target individual may select. For a suggested family tree, the tree management engine 260 may also provide information on how the target individual is related to other individuals in the tree. In a manual tree building, a user may browse through public family trees and public individual entries in the genealogy data store 200 and individual profile store 210 to look for potential relatives that can be added to the user's family tree. The tree management engine 260 may automatically search, rank, and suggest individuals for the user to conduct manual review as the user makes progress in the front-end interface 250 in building the family tree.

As used herein, “pedigree” and “family tree” may be interchangeable and may refer to a family tree chart or pedigree chart that shows, diagrammatically, family information, such as family history information, including parentage, offspring, spouses, siblings, or otherwise for any suitable number of generations and/or people, and/or data pertaining to persons represented in the chart. U.S. Patent Publication Application No., entitled “Linking Individual Datasets to a Database,” US2021/0216556, published on Jul. 15, 2021, describes example embodiments of how an individual may be linked to existing family trees.

### Example Matched Segment Identification

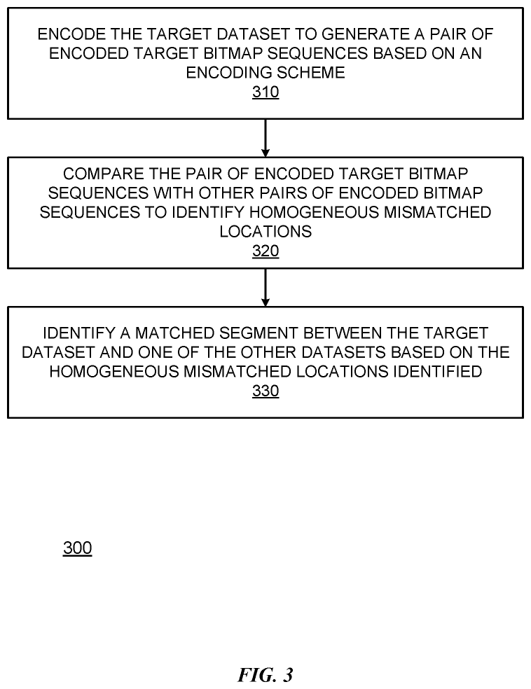

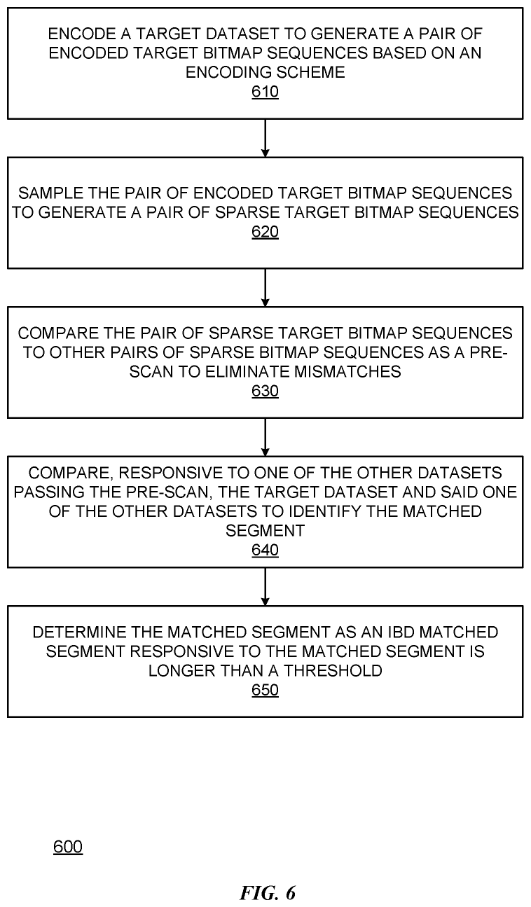

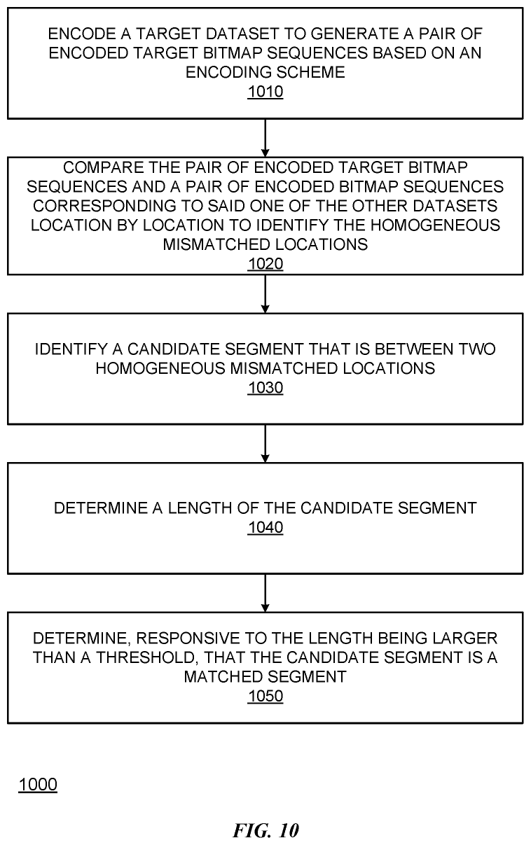

FIG. 3 is a flowchart depicting an example process 300 for identifying one or more segments of a target dataset that match segments of other datasets in a database, in accordance with some embodiments. For example, FIG. 3 may be part of a process or algorithm for fast genotyping for detecting Identity by descent (IBD) between pairs of genotyped individuals efficiently when the input data are large. Various steps in the process 300 may be processes that are performed by IBD estimation engine 225 or other engines in the computing server 130. Various processes may be implemented as one or more software algorithms. The software algorithm may be stored as computer instructions that are executable by one or more general processors (e.g., CPUs, GPUs). The instructions, when executed by the processors, cause the processors to perform various steps described. In various embodiments, one or more steps described may be skipped or changed. Steps described in FIG. 3 may also be combined with those in other figures, such as FIGS. 6 and 10. A computer-implemented process may be performed by the computing server 130, although the process may also be performed by another suitable computer.

The computer server 130 may encode 310 a target dataset to generate a pair of encoded target bitmap sequences based on an encoding scheme. A dataset may include a pair of data value sequences. For example, in the context of genetic data, the pair of data value sequences may be a diploid genotype sequence or a pair of phased haplotype sequences that are retrieved from genetic data store 205. The computing server 130 may store datasets of different users who took DNA tests and have the DNA data stored with the computing server 130. The target dataset may correspond to DNA data of a target individual, which may be phased or not phased. The computing server 130 may also store other datasets that correspond to DNA data of other individuals.

Table 1 and Table 2 below illustrate an example of how the pair of data value sequences in a genetic dataset may be represented. Each individual's phased genotype data may be represented as a list of ordered pairs of alleles corresponding to a list of biallelic SNPs. Each SNP in that list has one of its two alleles encoded as 0, and the other allele specified as being encoded as 1.

TABLE 1

| SNP | Allele represented as 0 | Allele represented as 1 |
|---|---|---|
| SNP #1 | A | G |
| SNP #2 | A | G |
| SNP #3 | A | G |
| SNP #4 | A | C |
| SNP #5 | A | G |
| SNP #6 | G | T |
| SNP #7 | A | G |
| SNP #8 | A | G |
| SNP #9 | A | G |
| SNP #10 | A | G |

For instance, if there are 10 SNPs in the list as shown in the table above, the individual's phased genotype data may be represented as the following:

TABLE 2

| | SNP | | | | | | | | | |
|---|---|---|---|---|---|---|---|---|---|---|
| | SNP | SNP | SNP | SNP | SNP | SNP | SNP | SNP | SNP | SNP |
| | #1 | #2 | #3 | #4 | #5 | #6 | #7 | #8 | #9 | #10 |
| Phased | (0, 0) | (0, 0) | (1, 1) | (0, 1) | (0, 1) | (1, 0) | (0, 0) | (1, 1) | (0, 0) | (0, 0) |
| genotype | | | | | | | | | | |

Note that the values are ordered, and (0,1) is not the same data as (1,0). A sequence of values (ϵ{0,1}) in either the first or second position in the data is referred to as phased haplotype data (or just “haplotype”). For instance, there is a haplotype <1,0,0,1,0> in the above table that is the allele in the first position for SNPs #3-#7 inclusive. There is another haplotype for that same range of SNPs, which represents the alleles in the second position. The other haplotype is <1,1,1,0,0>.

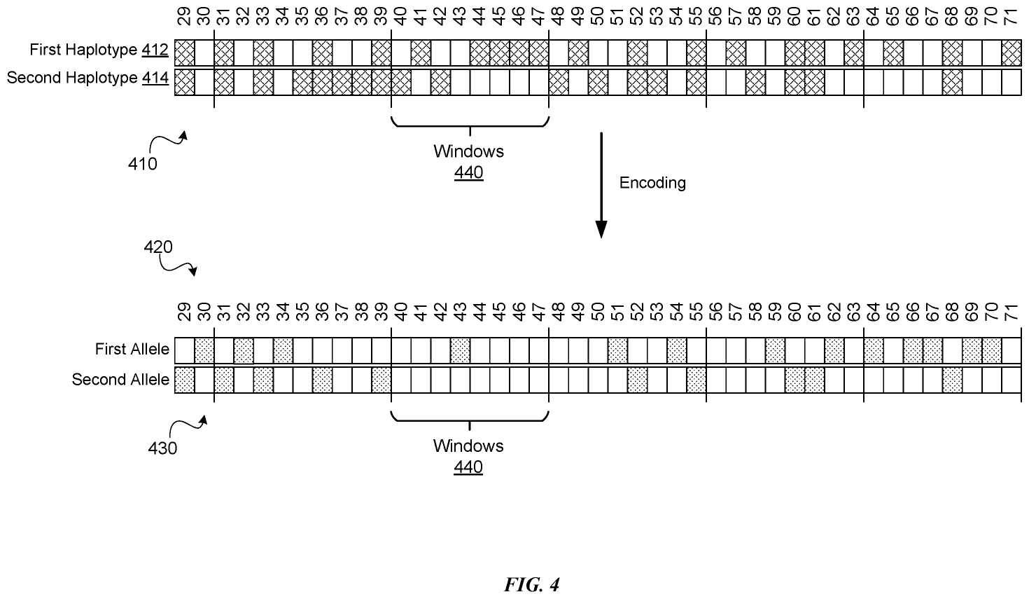

FIG. 4 is a conceptual diagram illustrating an example encoding scheme that may be used to encode a genetic dataset 410 to generate a pair of encoded bitmap sequences 420 and 430, in accordance with some embodiments. Since the encoding scheme described in FIG. 4 is different from the data representation illustrated in Tables 1 and 2, the generation of the bitmap sequences may also be referred to as re-encoding as Tables 1 and 2 may illustrate a first encoding scheme and the encoding scheme in FIG. 4 represents a second encoding scheme. The genetic dataset 410 is a diploid dataset that may or may not be phased. The genetic dataset 410 includes a pair of data value sequences, which may take the form of the first haplotype sequence 412 and the second haplotype sequence 414. Although in FIG. 4 the genetic dataset 410 is illustrated as phased data, the pair of data value sequences may also be unphased diploid genotype data generated by a DNA sequencer.

The genetic dataset 410 may carry allele values of different genomic positions. For example, the genetic dataset 410 may take the form of a genotype dataset or haplotype datasets retrieved from the genetic data store 205 and include various characteristics discussed above in association with the genetic data store 205 and the genetic data extraction service server 125. The genetic dataset 410 shown in FIG. 4 may be an abbreviated version, showing position 29 to position 71. An actual version of the genetic dataset 410 may include millions of positions or even billions of positions. While the position numbering of the genetic dataset 410 is consecutive, the position numbering does not necessarily correspond to the actual consecutive genomic position in a chromosome. For example, the positions in the genetic dataset 410 may only be SNP positions of interest. While at a given position, the possible nucleotide base value may be A, T, C, or G, the genetic dataset 410 may be stored in a simplified form that represents the major allele and the minor allele. For example, the genetic dataset 410 may carry allele information by taking a first value (e.g., 1, represented as a white block in FIG. 4) to represent the major allele and by taking a second value (e.g., 0, represented as a shaded block in FIG. 4) to represent the minor allele. Each data value sequence (e.g., the first haplotype 412 or the second haplotype 414) may have its own value at a given position. The genetic dataset 410 may be divided in windows 440 that allow the computing server 130 to better manage its data. For example, each window 440 in the example genetic dataset 410 shown in FIG. 4 has 8 positions. This allows the computing server 130 to store the values in a single window of one of the haplotypes by bits and bytes. For example, each position is a bit and the window of 8 positions can be stored as a byte.

In some embodiments, the major allele and the minor allele at a given position may be defined systematically or arbitrarily. For example, the computing server 130 may randomly pick one of the alleles as the major and other values as minor. In other embodiments, the computing server 130 may conduct statistics of allele values in a population of data. At a given position, if most of the population has the allele at the position, the allele will be considered the major allele. Other ways to decide on major or minor alleles may also be possible.

The encoding scheme may define encoding values based on homogeneity between the pair of data value sequences. Homogeneity may refer to a pair of data sequences having the same value at a given position. The pair of encoded target bitmap sequences includes a first encoded target bitmap sequence that encodes a first type of homogeneous locations and a second encoded target bitmap sequence that encodes a second type of homogeneous locations. In the context of DNA data such as genotype data, the homogeneity corresponds to the homozygosity between the pair of DNA sequences. For example, the first type of homogeneous locations corresponds to homozygous major alleles and the second type of homogeneous locations corresponds to homozygous minor alleles. Using DNA data as an example, the encoding scheme defines that the first encoded bitmap sequence 420 has a first value (e.g., 1, represented as a shaded block in element 420 of FIG. 4) if the pair of data value sequences 412 and 414 are homozygous first allele (e.g., major allele) and has a second value otherwise (e.g., heterozygous or homozygous second allele). The encoding scheme defines that the second encoded bitmap sequence 430 has the first value (e.g., 1, represented as a shaded block in element 430 of FIG. 4) if the pair of data value sequences are homozygous second allele (e.g., minor allele) and has the second value otherwise (e.g., heterozygous or homozygous first allele).

To further illustrated, FIG. 4 is described in more detail. Phased genotype data 412 and 414 are represented. At each position of sequence 412 or 414, a shaded block represents one allele and the white block represents the other allele. Each biallelic SNP is encoded arbitrarily (without a loss of generality, white blocks could represent the major allele and shaded blocks could represent the minor allele). For example, individual A with the genotype shown in FIG. 4 is homozygous for the major allele at position 34, heterozygous at position 35, and homozygous for the minor allele at position 36. Each position (SNP position) is numbered in order and lines are drawn between windows of 8 SNPs.

In order to perform scanning of genotypes more efficiently, the computing server 130 may encode the phased genotype data 412 and 414 for each individual in the genetic data store 205 into a pair of homozygosity bitmap sequences. An example of the bitmap pair is shown as elements 420 and 430. In some embodiments, the computing server 130 may store millions of individuals' genetic data. In some embodiment, in addition to each individual's genotype data and phased genotype data, the individual genetic data may be additionally stored as two homozygosity bitmap sequences that are encoded as integers. For example, homozygous bitmap sequences 420 and 430 for individual A are shown in FIG. 4. The first homozygosity bitmap sequence 420 for individual A is encoded as a value that is graphically illustrated as shaded if individual A is homozygous for the major allele (e.g., the first allele value) at the corresponding position. The second homozygous bitmap sequence 430 for individual A is shaded if individual A is homozygous for the minor allele (e.g., the second allele value) at the corresponding position.

The encoding for the window that includes position 48 through position 55 is discussed in further detail as an example. Position 48 is heterozygous. Hence, both bitmap sequences 420 and 430 are encoded with white blocks (e.g., second value, or 0) at position 48. The same encoding is performed for positions 49 and 50 as both positions are heterozygous. For position 51, it is a homozygous position with the major allele (first allele). Hence, the bitmap sequence 420 is encoded with a shaded block (e.g., first value, or 1) at position 51 but the bitmap sequence 430 is still encoded with a white block because, for the bitmap sequence 430, either a heterozygous position or a homozygous position with the major allele is encoded with a white block. For the bitmap sequence 430, only a homozygous position with the minor allele is encoded with a shaded block. For positions 60 and 61, both positions are homozygous with the minor allele (second allele). Hence, the bitmap sequence 430 is encoded with shaded blocks at both positions 60 and 61. The bitmap sequence 420 is encoded with white blocks at positions 60 and 61 because either a heterozygous position or a homozygous position with the minor allele is encoded with a white block in bitmap 420. For position 62, it is a homozygous position with the major allele. Hence, the bitmap sequence 420 is encoded with a shaded block at position 62 and the bitmap sequence 430 is encoded with a white block at position 62. Position 63 is heterozygous. Hence, both bitmap sequences 420 and 430 are encoded with white blocks. As the encoding scheme is based on homozygosity, the genetic dataset 410 may be phased or not phased.

Referring back to FIG. 3, the computing server 130 may compare 320 the pair of encoded target bitmap sequences with other pairs of encoded bitmap sequences to identify homogeneous mismatched locations. The encoded target bitmap sequences may be encoded from the genetic data of a target individual. Other pairs of encoded bitmap sequences may be encoded from the genetic data of other individuals in the database of computing server 130 using the encoding scheme. A homogeneous mismatched location is a location where the target dataset and the other dataset in comparison are both homogeneous but have different types of homogeneity at the location. For example, in the context of DNA data, a homogeneous mismatched location is a homozygous mismatched location. A homozygous mismatched location may refer to a location where the two individuals in comparison are both homozygous but have different alleles. For example, one individual may be homozygous with the major allele. Another individual may be homozygous with the minor allele.

The computing server 130 may identify 330 a matched segment between the target dataset and one of the other datasets based on the homogeneous mismatched locations identified. The matched segment may be a segment that is contained within two homogeneous mismatched locations. In the context of DNA data, the matched segment may be an identity by descent (IBD) segment between two individuals. In this context, a matched segment does not necessarily require that both datasets have absolutely identical segments. Instead, a matched segment may be a segment that does not contain a homozygous mismatched location. For example, a matched segment may contain heterozygous mismatches or a position that is homozygous for one individual and heterozygous for another individual. In some embodiments, the longest matched segment in a genetic locus may be a segment between two homozygous mismatched locations.

Detection of an IBD segment may be performed by a conventional process that is commonly referred to as GERMLINE, which may be performed by the IBD estimation engine 225. The IBD estimation engine 225, typically using a hash table, finds a small segment (called a seed range of match) of DNA identity between two individuals' genotype data, and scans the genotype data of each such pair of individuals to find Mendelian errors (e.g., homozygous mismatches) that delimit shared segments. The IBD estimation engine 225 returns the shared segments that are greater than or equal to a threshold length that is previously specified.

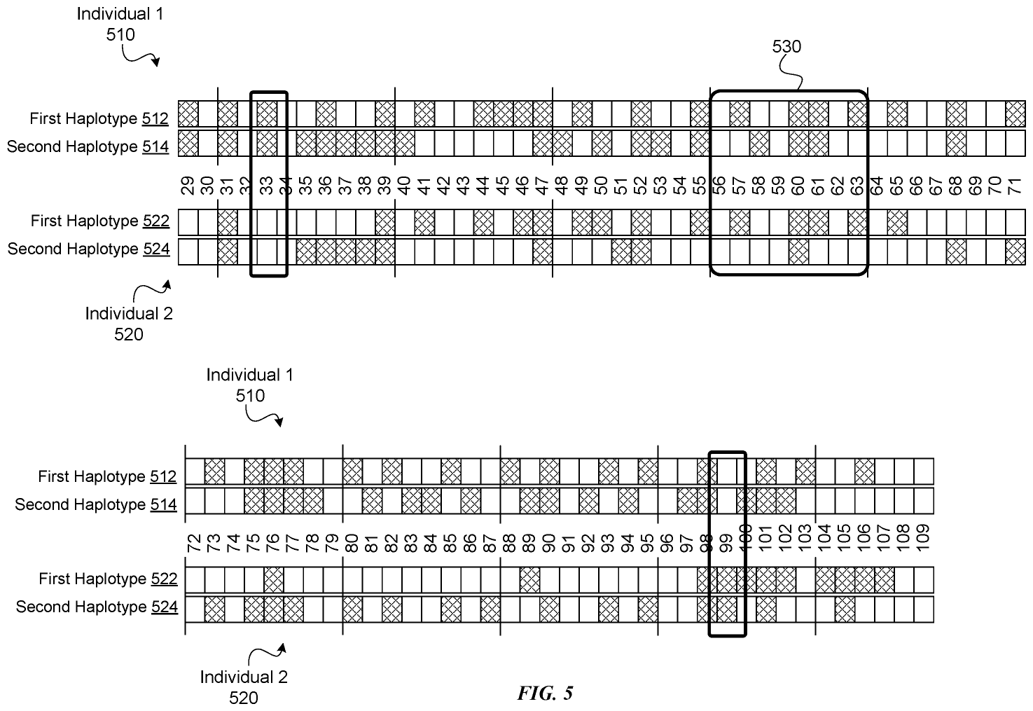

FIG. 5 is a conceptual diagram illustrating the detection of an IBD segment shared between two individuals. The first pair of data value sequences represent the phased genotype data of the first individual 510 and includes the first haplotype 512 and the second haplotype 514. Both phased genotype data are broken at positions 71 and 72 due to the spacing of the figure. The second pair of data value sequences represents the phased genotype data of the second individual 520 and includes the first haplotype 522 and the second haplotype 524. The allele representation scheme is the same as that in FIG. 4. The IBD estimation engine 225 uses a hash table to group pairs of individuals that share an identical window-haplotype. For example, the haplotype for each window (e.g., an 8-bit value) may be hashed. These two individuals A and B share one haplotype in the boxed window 530 (SNPs 56-63 inclusive). The haplotype 512 of the first individual and the haplotype 522 of the second individual are identical and can be easily identified in the hash table. Note that haplotype 514 of the first individual and the haplotype 524 of the second individual are not a match. The seed range of match may be identified if at least one of the two haplotypes is a match.

The IBD estimation engine 225 identifies this seed match pair in the window 530 (since both have haplotypes that hash to the same hash table position) and tags it as the seed range of match. After a seed range of match is identified, the phased nature of the data may be ignored. The IBD estimation engine 225 in turn examines the phased genotype data around the seed window to measure the length of IBD—e.g., by finding the nearest points on either side where the two individuals do not share an allele (they have a Mendelian error, a homozygous mismatch). For example, the IBD estimation engine 225 scans to the left and right (upstream/downstream) of the seed match to find the nearest SNP where the two individuals in question do not share an allele (e.g., one has data (0,0) and the other has (1,1) for one SNP). In FIG. 5, these are at positions 33 and 99. As such, an IBD segment may be reported as SNPs 34-98 inclusive (unless that segment fails some other criteria, such as not being long enough to be considered significant). After the segment endpoints are identified, the computing server 130 returns the segment if it is of some user-specified minimum length (e.g., 5 cM) or longer. The genotype data for two individuals can be compared one SNP site at a time. If haplotypes are encoded as integers, multiple SNPs can be compared simultaneously using a series of bitwise operations.

### Example Pre-Scan Process Using Bitmap Encoding

FIG. 6 is a flowchart illustrating an example fast IBD identification process 600 using a pre-scan step that compares bitmap sequences to eliminate mismatches, in accordance with some embodiments. The process 600 may be an example of process 300. In various embodiments, one or more steps described may be skipped or changed. The process 600 is a process of fast genotyping for detecting IBD shared by two individuals in an efficient manner, according to some embodiments. The process re-encodes the genotype data to improve the scanning speed and scans on a carefully-constructed subset of the re-encoded data to eliminate false matches. The process may break up the seed segment over a larger range for the purpose of only finding IBD of a significant length. The process may also first load the re-encoded data for a large set of genotype data (e.g., those of hundreds of thousands or millions of individuals), then act as a constantly running real-time server for computing IBD against arbitrary query genotype data.

The computing server 130 may encode 610 a target dataset to generate a pair of encoded target bitmap sequences based on an encoding scheme. The encoding scheme may be the encoding scheme that is explained in FIG. 4.

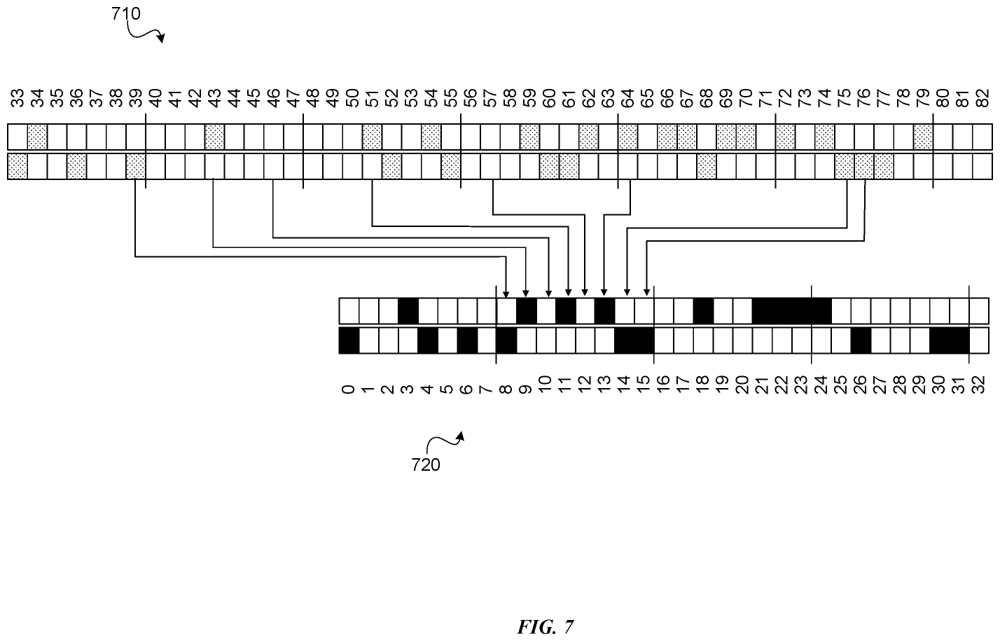

The computing server 130 may sample 620 the pair of encoded target bitmap sequences to generate a pair of sparse target bitmap sequences. A sparse bitmap sequence may take the form of an ordered subset of the full homozygosity bitmap sequence. FIG. 7 is a conceptual diagram illustrating an example sampling process for turning a pair of bitmap sequences 710 into a pair of sparse bitmap sequences 720, in accordance with some embodiments. The computing server 130 may sample some of the locations of the pair of bitmap sequences 710 and generate a shorter pair of bitmap sequences that may be referred to as a pair of sparse bitmap sequences 720. The sparse bitmap sequences 720 are shorter than the bitmap sequences 710 because only some of the locations in the bitmap sequences 710 are sampled to the sparse bitmap sequences 720. While the examples shown in FIG. 7 the sequences are shorter than 100 bases, in practice for some embodiments the bitmap sequences 710 may be millions of bases long and the sparse bitmap sequences 720 may still be millions of bases long (despite shorter than bitmap sequences 710) or hundred of thousands of bases long. The numerical positions of the bitmap sequences 710 and the sparse bitmap sequences 720 do not need to correspond. The computing server 130 stores a mapping of the positions. For example, position 39 in the bitmap sequences 710 is sampled at position 8 in the sparse bitmap sequences 720. The sparse bitmap sequences 720 may also be divided in windows but the window lengths in the bitmap sequences 710 and in the sparse bitmap sequences 720 are necessarily related. In some embodiments, both types of sequences use the window length of 8 so that each window may carry a byte of data.

In sampling various positions, the encoded homozygosity values in the bitmap sequences 710 are carried to the sparse bitmap sequences 720. For example, positions 39, 43, 47, 51, 57, 64, 75, and 76 in the bitmap sequences 710 are sampled to positions 8 through 15 in the sparse bitmap sequences 720. The homozygosity values in position 39 in the bitmap sequences 710 are carried to position 8 in the sparse bitmap sequences 720. Hence, the lower sequence has the value 1. Likewise, the homozygosity values in position 43 in the bitmap sequences 710 are carried to position 9 in the sparse bitmap sequences 720. Hence, the upper sequence has the value 1. In position 46 in the bitmap sequences 710, the homozygosity values are zero for both sequences. As such, the values are zero at position 10 in the sparse bitmap sequences 720.

In some embodiments, the bitmap sequences 710 may be sampled in any suitable manner, arbitrarily or patterned, evenly spaced or not. For example, in some embodiments, the selection of the positions to be sampled to form the sparse bitmap sequences 720 may be completely arbitrary. In other embodiments, some rules may be introduced in the selection of positions in addition to or in alternative to random sampling. For example, the computing server 130 may review a large collection of genetic datasets (e.g., over a million datasets in some embodiments) to determine what positions are likely to contain homozygous mismatches. Those positions may be selected by the computing server 130 as key positions to sample. Other rules may also be used in sampling. For example, certain gene locations or SNP locations are known to be related to certain phenotypes (e.g., certain traits of a person or certain diseases). Those positions may also be selected for the sampling process. Alternatively, or additionally, the computing server 130 may choose the SNPs that will go into the sparse bitmaps based on empirical data and properties of those SNPs. By way of example, the computing server 130 chooses the SNPs that have the highest minor allele homozygosity rate from a large sample of data. The computing server 130 may also choose the SNPs based on minor allele frequency, genotype or imputation error frequency or other criteria.

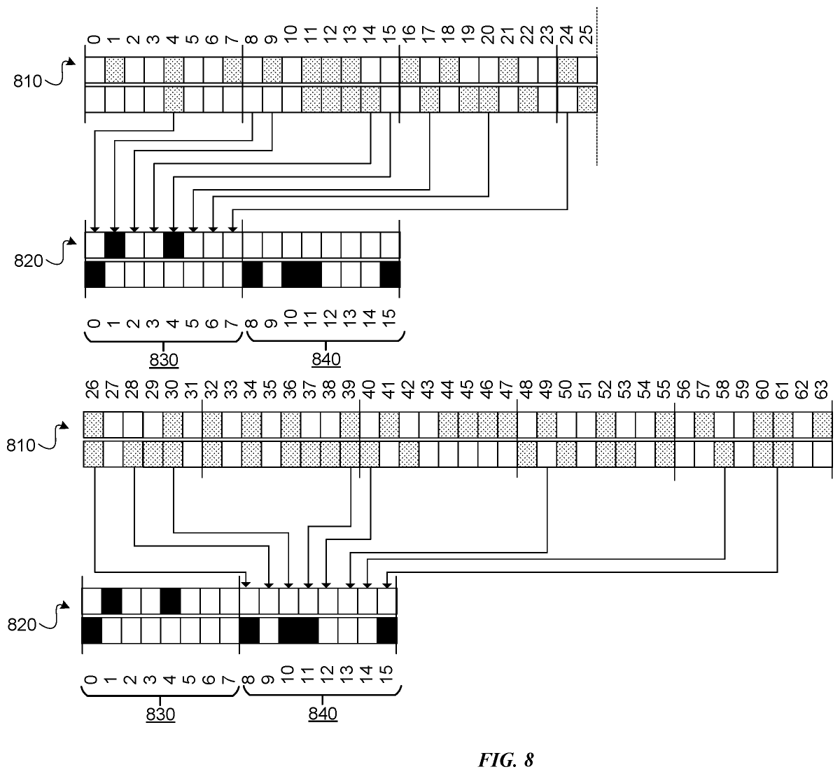

In various embodiments, the frequency of sampling may also be completely arbitrary, be partially arbitrary with some rules, or strictly follow some rules. In some embodiments, a window in the sparse bitmap sequences 720 represents a fixed length (or approximately fixed length where the variations in lengths are less than 50%). FIG. 8 is a conceptual diagram illustrating an example way of sampling such that each window in the sparse bitmap sequences 820 similarly represents an approximately fixed length in the bitmap sequences 810. In FIG. 8, the bitmap sequences 810 and sparse bitmap sequences 820 are separated into two sets due to the spacing of the drawing. In FIG. 8, the sparse bitmap sequences 820 includes two windows 830 and 840. The bases of the sparse bitmap sequences 820 in window 830 are sampled from positions 0 to 25 of the bitmap sequences 810. The bases of the sparse bitmap sequences 820 in window 840 are sampled from positions 26 to 61 of the bitmap sequences 810.

In some embodiments, the sampling frequency and the windows in the sparse bitmap sequences 820 may be related to the threshold length of a matched segment that is to be considered an IBD segment. For example, the computing server 130 may set the minimum IBD segment threshold. Segments that are shorter than the threshold, despite matching, may not be considered IBD segments. This requires two individuals to share DNA segments of sufficient length in order to be considered IBD related. In some embodiments, for example, the minimum IBD segment length threshold may be set at 5 centimorgans (cM) (or another suitable threshold), the computing server 130 may choose the size of a sparse homozygosity bitmap window to be 2.5 cM. As such, the windows 830 and 840 may each represent approximately 2.5 cM of bases in the unsampled bitmap sequences 810. The sampling frequency may be accordingly adjusted so that each window in the sparse bitmap sequences 820 is at least close to the chosen window length. In general, the size of a sparse homozygosity bitmap window may be chosen to be a portion (e.g., half) of the size or less of the minimum segment length. The numbers 5 and 2.5 cM are used as non-limiting examples only.

In the example shown in FIG. 8, each 8-SNP window of the sparse bitmap sequences 820 represents some amount of a genome, which may be measured in centimorgans. In this example, let it be 1 cM (instead of 2.5 cM in the previous paragraph). Suppose that SNPs 0-25 (inclusive) represent the same distance (e.g., if the computing server 130 included SNP number 26, the window would exceed 1 cM). The computing server 130 maps 8 of the 26 SNPs in that set to the first sparse SNP window 830. The choice may be based on minor allele frequency, minor allele homozygosity rate, or some other empirical measure, or it may be chosen randomly, uniformly, or by some other method. The next 36 SNPs (SNPs 26-61 inclusive) represent the next 1 cM of the genome. Another 8 of those SNPs are selected for the second sparse homozygosity bitmap window 840 and the selection process may continue across a whole chromosome of genomic data.

Referring back to FIG. 6, the computing server 130 may compare 630 the pair of sparse target bitmap sequences to other pairs of sparse bitmap sequences as a pre-scan to eliminate mismatches. The pair of sparse target bitmap sequences may correspond to an encoded and sampled genetic dataset of the target individual. Other pairs of sparse bitmap sequences may correspond to encoded and sampled genetic datasets of other individuals. In some embodiments, since the sparse bitmap sequences are shorter than the full bitmap sequences, comparing sparse bitmap sequences to identify mismatches such as homozygous mismatches can be significantly more efficient than using the full bitmap sequences.

By way of example, if two individuals' genotype data have a homozygous mismatch in the sparse bitmap sequences, the two individuals will also have a homozygous mismatch in the full bitmap sequences or the full genotype datasets. As such, this subset can act as a filter to eliminate seed matches from consideration by comparing fewer data than with the full bitmap sequences.

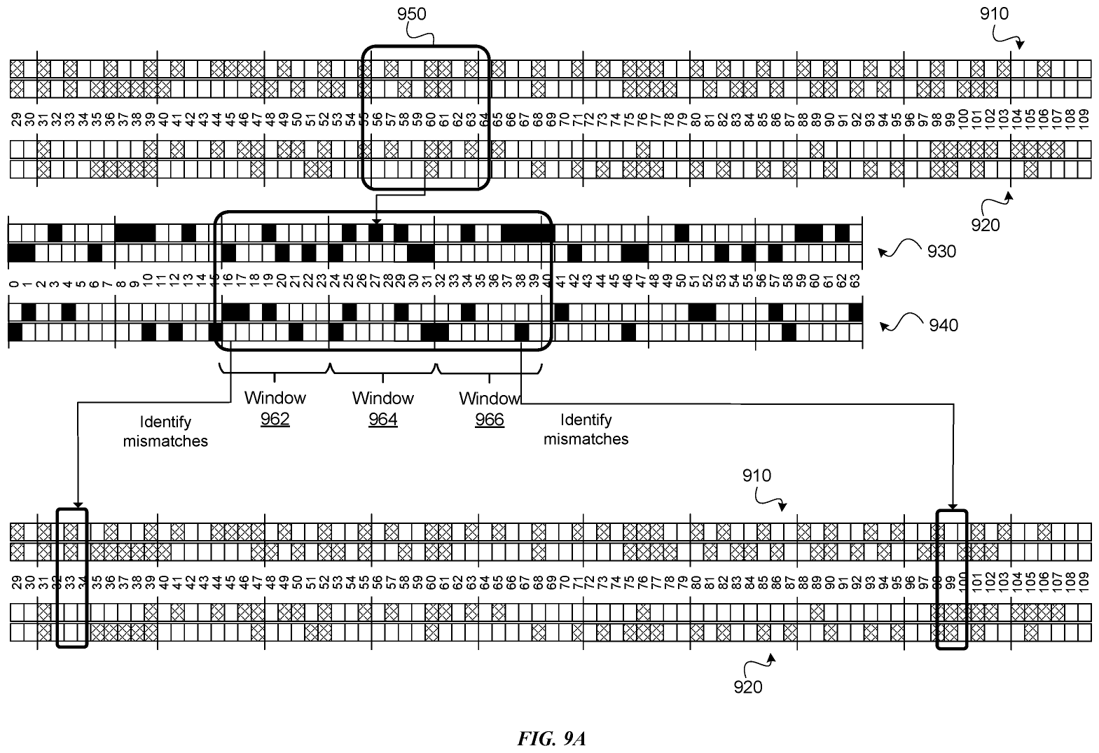

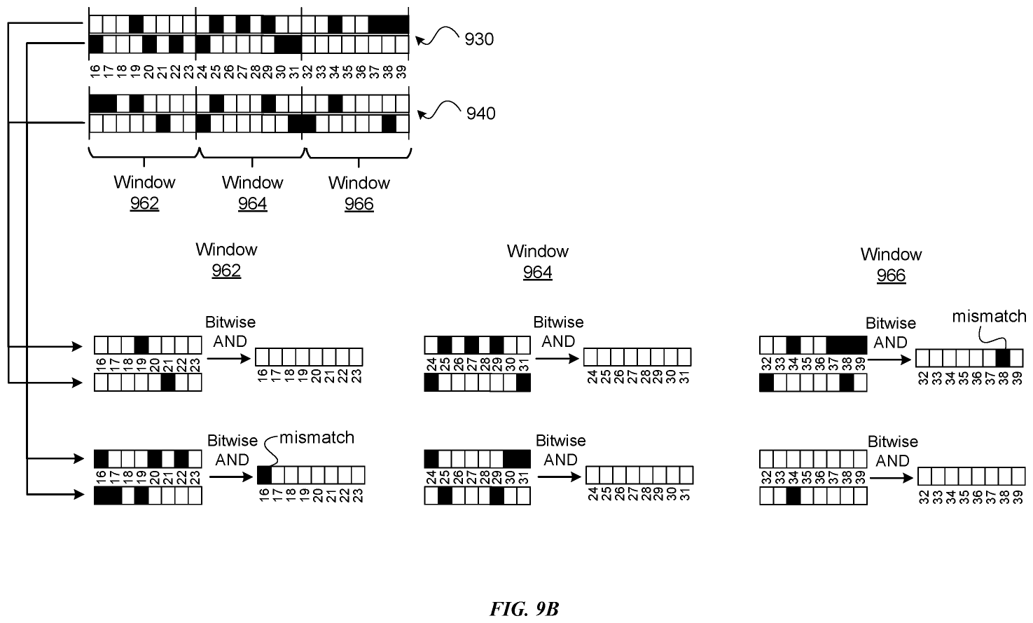

FIG. 9A is a conceptual diagram illustrating an example process for comparing sparse bitmap sequences of two individuals to identify the homozygous mismatched locations of the two individuals, in accordance with some embodiments. In FIG. 9A, the pair of data value sequences 910 for a first individual and the pair of data value sequences 920 for a second individual are shown. The data value sequences 910 and data value sequences 920 are genotype or phased genotype sequences before applying the encoding scheme discussed in FIG. 4. The data value sequences 910 and data value sequences 920 may be encoded using the encoding scheme discussed in FIG. 4 and generate two pairs of full homozygosity bitmap sequences, which are not shown in FIG. 9A. From the two pairs of full homozygosity bitmap sequences, two pairs of sparse bitmap sequences may be generated using step 620. The pair of sparse bitmap sequences 930 corresponds to the first individual (e.g., the target individual) and the pair of sparse bitmap sequences 940 corresponds to the second individual.

In step 630, the computing server may compare the pair of sparse target bitmap sequences corresponding to the target individual (e.g., the sparse bitmap sequences 930) to another pair of sparse bitmap sequences (e.g., the sparse bitmap sequences 940) as a pre-scan to eliminate mismatches. For example, the step 630 may include identifying a seed range of match between the target dataset (e.g., data value sequences 910) and another dataset (data value sequences 920) of the second individual. For example, in the window 950, the top haplotype of the data value sequences 910 and the top haplotype of the data value sequences 920 are identified and can be easily identified in a hash table that stores hashed values of SNPs for each window for sequences 910 and 920.

The computing server 130 may compare the pair of sparse target bitmap sequences with one of the other pairs of sparse bitmap sequences upstream and downstream of the seed range to identify the homogeneous mismatched locations. For example, in identifying that the window 950 contains a matched haplotype between two individuals, the computing server 130 may determine that one or more corresponding positions in window 950 now are within the window 964 in the sparse bitmap sequences 930 and 940. In other words, the middle window 964 of the three marked windows 962, 964, and 966 in the sparse bitmap sequences is the one that corresponds to the seed match highlighted in the phased genotype data 910 and 920. The computing server 130 may compare the sparse bitmap sequences between the two individuals upstream and downstream of the seed range, such as by examining the bitmaps in windows 962, 964, and 966.

The comparing step 630 may include comparing the first encoded target bitmap sequence (e.g., the top sequence of the pair of the sparse bitmap sequences 930) that encodes the first type of homozygous locations of the target dataset (e.g., the homozygous major allele of the data value sequences 910) to the second encoded bitmap sequence of another pair (e.g., the bottom sequence of the pair of the sparse bitmap sequences 940). The second encoded bitmap sequence encodes the second type of homozygous locations (e.g., the homozygous minor allele of the data value sequences 920). The computing server 130 may also compare the bottom sequence of the pair of the sparse bitmap sequences 930 (encoding minor allele homozygosity of the first individual) with the top sequence of the pair of the sparse bitmap sequences 940 (encoding major allele homozygosity of the second individual). The scan between two individuals can be performed faster because the computing server 130 needs only two comparisons in each window. The computing server 130 identifying a common location that indicates the target dataset and the other dataset in comparison are both homogeneous but have different alleles.

FIG. 9B is a conceptual diagram illustrating a comparison process for comparing two bitmap sequences to identify homozygous mismatch, in accordance with some embodiments. In FIG. 9B, the two pairs of sparse bitmap sequences 930 and 940 in the windows 962, 964, and 966 are reproduced from FIG. 9A. Any two sequences may be compared using a single bitwise AND operation. If the operation returns anything other than zero, there is a homozygous mismatch somewhere in the window. Hence, for example, the bitwise AND operation results can be accumulated by an adder and any final value that is larger than zero indicates that there is a homozygous mismatch somewhere in the window. Since processors and digital circuits often have built-in circuitry such as AND gates that can perform bitwise AND operation and add operation extremely quickly, the process illustrated in FIG. 9B increases the speed of identification of IBD matched segments significantly.

By way of example, in FIG. 9B, the top sequence of the pair of the sparse bitmap sequences 930 (encoding major allele homozygosity of the first individual) is compared with the bottom sequence of the pair of the sparse bitmap sequences 940 (encoding minor allele homozygosity of the second individual) using pairwise AND operations for each window. Likewise, the bottom sequence of the pair of the sparse bitmap sequences 930 (encoding minor allele homozygosity of the first individual) is compared with the top sequence of the pair of the sparse bitmap sequences 940 (encoding major allele homozygosity of the second individual) using pairwise AND operations for each window. The AND operation only generates 1 when both input values are 1. If either input value is 0, the AND operation will generate 0. For window 962, the pairwise AND operations quickly determine that there is a homozygous mismatch at position 16. For window 964, the pairwise AND operations quickly determine that all values are zero and there is no homozygous mismatch. For window 966, the pairwise AND operations quickly determine that there is a homozygous mismatch at position 38. Referring back to FIG. 9A, after the mismatch positions are located, the computing server 130 may trace back to the genotype or haplotype datasets 910 and 920 to identify the corresponding homozygous mismatch positions, which are at positions 33 and 99 in FIG. 9A.

While FIG. 9B is illustrated by using sparse bitmap sequences, full bitmap sequences such as those illustrated in sequences 430 may also be compared using the process illustrated in FIG. 9B.

In some embodiments, comparing the pair of encoded bitmap sequences for the target induvial with another pair of encoded bitmap sequences upstream and downstream of the seed range may stop at a threshold range. Each seed match location corresponds to a window in the sparse homozygosity bitmap (the window that contains the same or surrounding SNPs of the seed match). Each window in the sparse bitmap sequences may be set to represent about half of the length required for the minimum threshold of length in order for a segment to be regarded as a match segment (an IBD segment). For example, if the threshold is 5 cM or more, each window in the sparse bitmap sequences 930 and 940, such as windows 962, 964, and 966, may represent a length of 2.5 cM. If, in the sparse bitmap sequences, the seed window 964 and the adjacent windows 962 and 966 on either side of the seed window 964 all contain a homozygous mismatch, the seed match cannot be part of an IBD segment of sufficient length. The computing server 130 can determine that the candidate segment fails the pre-scan and move on to another candidate. This is because every 5 cM or more long matched segment contains at least one entire fixed position 2.5 cM window. Hence, two consecutive sparse windows that contain a mismatch will not correspond to a matched segment of sufficient length. If two consecutive sparse windows do not contain any homozygous mismatch, the pre-scan is passed for those two or more windows.

Referring back to FIG. 6, the computing server 130 may compare 640, responsive to two datasets in comparison passing the pre-scan, the target dataset and said one of the other datasets to identify the matched segment. The comparison in step 640 may be based on full, unsampled data values. The data values may be from the genotype or haplotype data 910 and 920 or from full bitmap sequences. Since the sparse bitmap sequences only include certain sampled positions, the homozygous mismatch positions found (if any) by comparing the sparse bitmap sequences may not be the shortest separated homozygous mismatch positions. There may still be one or more homozygous mismatch positions that are not sampled and thus missed by the computing server 130 when examining the sparse bitmap sequences. As such, the computing server 130 compares the full, unsampled data values to find one or more mismatches.

In some embodiments, since the DNA data a target individual (e.g., an individual who recently took a DNA test) may be compared to millions of users whose DNA data are stored in the computing server 130, the target dataset may need to be compared to millions of other datasets. Since the sparse bitmap sequences are shorter than the full sequences and the comparison may be performed efficiently using bitwise AND operations, the pre-scan step described in step 630 may speed up the entire process of IBD identification because a large number of mismatched candidates are eliminated.

The computing server 130 may determine 650 the matched segment as an IBD matched segment responsive to the matched segment is longer than a threshold. For example, after a matched segment is detected in step 640, the computing server 130 may compare the segment to a threshold length. If the computing server 130 determines that the segment is longer than the threshold length, the computing server 130 may determine that there is an IBD match. In some cases, the computing server 130 may also notify the IBD estimation result to the target individuals using a graphical user interface. The IBD relationship may be stored in the individual profile store 210 and may be used to suggest relatives for the target individual to build a family tree using engines such as the tree management engine 260.

### Example Matched Segment Identification Process

FIG. 10 is a flowchart depicting an example process 1000 for determining a matched segment, in accordance with some embodiments. The process 1000 may be an example of process 300. In various embodiments, one or more steps described may be skipped or changed. The process 1000 is a process of fast genotyping for detecting IBD shared by two individuals in an efficient manner, according to some embodiments. In some embodiments, the process re-encodes the genotype data to improve the scanning speed without the need of running a phasing algorithm to convert genotype to haplotypes. The skipping of phasing algorithms, such as those performed by the phasing engine 220, can increase the speed of the overall process as the phasing algorithm can be a complex and relatively time-consuming algorithm.

The computing server 130 may encode 1010 a target dataset to generate a pair of encoded target bitmap sequences based on an encoding scheme. The encoding scheme may be the encoding scheme that is explained in FIG. 4.

The computing server 130 may compare 1020 the pair of encoded target bitmap sequences and a pair of encoded bitmap sequences corresponding to said one of the other datasets location-by-location to identify the homogeneous mismatched locations. In some embodiments, the pair of encoded target bitmap sequences are generated from unphased data of the target dataset so phasing is not required. In some embodiments, the comparison step 1020 may use a process that is similar to any conventional process run by the IBD estimation engine 225 such as GERMLINE or JERMLINE. In some embodiments, the location-by-location comparison is by using full bitmap sequences (instead of sparse) and bitwise AND operations explained in FIG. 9B may be used to speed up the process. Conventionally, using unphased genotype datasets to run IBD estimation algorithms such as GERMLINE or JERMLINE is extremely slow. The use of the encoding to generate homozygosity bitmap sequences and the use of bitwise AND operations significantly speeds up the process.

The computing server 130 may identify 1030 a candidate segment that is between two homogeneous mismatched locations. The computing server 130 may determine 1040 a length of the candidate segment. The computing server 130 may determine 1050, responsive to the length being larger than a threshold, that the candidate segment is a matched segment. In some cases, the computing server 130 may also notify the IBD estimation result to the target individuals using a graphical user interface. The IBD relationship may be stored in the individual profile store 210 and may be used to suggest relatives for the target individual to build a family tree using engines such as the tree management engine 260.

### Experimental Results

Table 3 below shows experimental data comparing the performance of a baseline conventional IBD estimation algorithm GERMLINE and a novel process 600 in estimating IBD segments for one of the chromosomes. 10,000 individuals' datasets were run. Table 3 shows that the process 600 in accordance with some embodiments improves the runtime speed and memory usage significantly.

TABLE 3

| | | | System | Memory |
|---|---|---|---|---|
| | | User time | time | use |
| METHOD | Wall-clock | (seconds) | (seconds) | (kbytes) |
| GERMLINE | 11:17.9 | 277.71 | 23.7 | 1472780 |
| Process 600 | 01:20.3 | 78.29 | 1.87 | 577516 |

Table 4 below shows experimental data comparing the performance of various processes, including a conventional IBD estimation algorithm and various novel processes described in this disclosure in accordance with various embodiments. Again, the processes were run for estimating IBD segments for one of the chromosomes. 10,000 individuals' datasets were run. PTER are methods that use homozygous bitmap sequences. PTER-unphased is a version that uses the homozygous bitmap sequences to process unphased data. Table 4 shows that the processes in accordance with various embodiments improve the runtime speed and memory usage significantly.

TABLE 4

| | | | System | Memory |
|---|---|---|---|---|
| | | User time | time | use |
| METHOD | Wall-clock | (seconds) | (seconds) | (kbytes) |
| GERMLINE | 11:17.9 | 277.71 | 23.7 | 1472780 |
| PTER | 01:20.3 | 78.29 | 1.87 | 577516 |
| PTER-unphased | 03:48.0 | 227.31 | 0.7 | 218600 |
| PTER (16 threads) | 00:23.1 | 95.45 | 2.12 | 578112 |
| PTER-unphased | 00:28.4 | 264.98 | 3.1 | 230068 |
| (16 threads) | | | | |

### Computing Machine Architecture

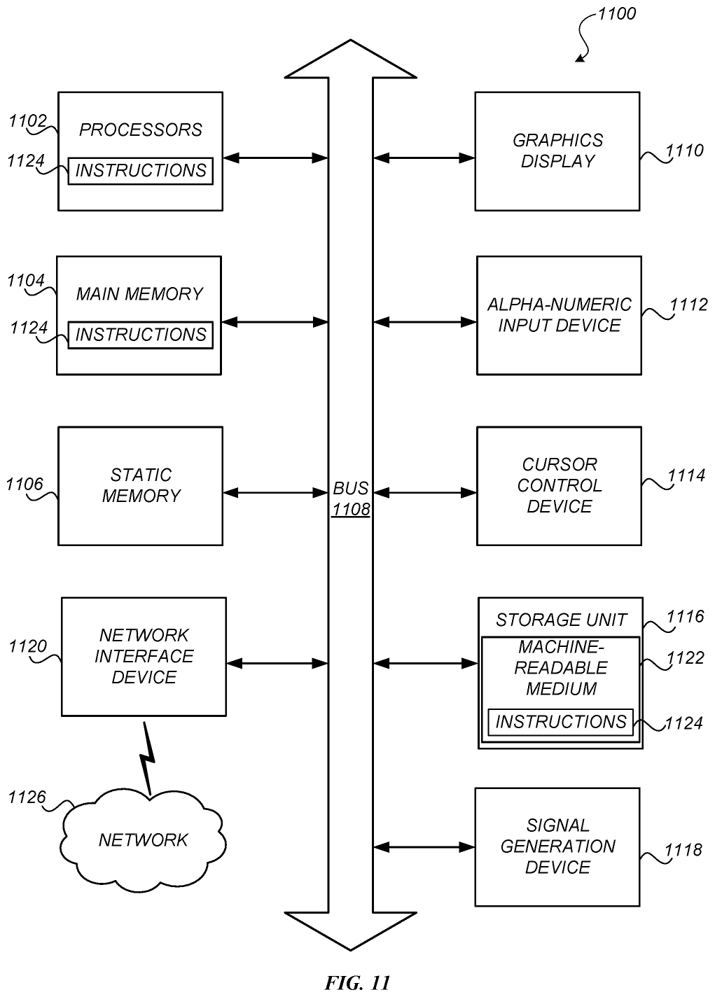

FIG. 11 is a block diagram illustrating components of an example computing machine that is capable of reading instructions from a computer-readable medium and execute them in a processor (or controller). A computer described herein may include a single computing machine shown in FIG. 11, a virtual machine, a distributed computing system that includes multiples nodes of computing machines shown in FIG. 11, or any other suitable arrangement of computing devices.

By way of example, FIG. 11 shows a diagrammatic representation of a computing machine in the example form of a computer system 1100 within which instructions 1124 (e.g., software, source code, program code, expanded code, object code, assembly code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 11 may correspond to any software, hardware, or combined components shown in FIGS. 1 and 2, including but not limited to, the client device 110, the computing server 130, and various engines, interfaces, terminals, and machines shown in FIG. 2. While FIG. 11 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 1124 that specify actions to be taken by that machine. Further, while only a single machine is illustrated, the term “machine” and “computer” may also be taken to include any collection of machines that individually or jointly execute instructions 1124 to perform any one or more of the methodologies discussed herein.

The example computer system 1100 includes one or more processors 1102 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 1100 may also include a memory 1104 that store computer code including instructions 1124 that may cause the processors 1102 to perform certain actions when the instructions are executed, directly or indirectly by the processors 1102. Instructions can be any directions, commands, or orders that may be stored in different forms, such as equipment-readable instructions, programming instructions including source code, and other communication signals and orders. Instructions may be used in a general sense and are not limited to machine-readable codes. One or more steps in various processes described may be performed by passing through instructions to one or more multiply-accumulate (MAC) units of the processors.

One and more methods described herein improve the operation speed of the processors 1102 and reduces the space required for the memory 1104. For example, the database processing techniques and machine learning methods described herein reduce the complexity of the computation of the processors 1102 by applying one or more novel techniques that simplify the steps in encoding sequences and comparing sequences and a large amount of data for the processors 1102. The algorithms described herein also reduces the size of the models and datasets to reduce the storage space requirement for memory 1104.

The performance of certain of the operations may be distributed among the more than processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations. Even though in the specification or the claims may refer some processes to be performed by a processor, this should be construed to include a joint operation of multiple distributed processors.

The computer system 1100 may include a main memory 1104, and a static memory 1106, which are configured to communicate with each other via a bus 1108. The computer system 1100 may further include a graphics display unit 1110 (e.g., a plasma display panel (PDP), a liquid crystal display (LCD), a projector, or a cathode ray tube (CRT)). The graphics display unit 1110, controlled by the processors 1102, displays a graphical user interface (GUI) to display one or more results and data generated by the processes described herein. The computer system 1100 may also include alphanumeric input device 1112 (e.g., a keyboard), a cursor control device 1114 (e.g., a mouse, a trackball, a joystick, a motion sensor, or other pointing instrument), a storage unit 1116 (a hard drive, a solid state drive, a hybrid drive, a memory disk, etc.), a signal generation device 1118 (e.g., a speaker), and a network interface device 1120, which also are configured to communicate via the bus 1108.

The storage unit 1116 includes a computer-readable medium 1122 on which is stored instructions 1124 embodying any one or more of the methodologies or functions described herein. The instructions 1124 may also reside, completely or at least partially, within the main memory 1104 or within the processor 1102 (e.g., within a processor's cache memory) during execution thereof by the computer system 1100, the main memory 1104 and the processor 1102 also constituting computer-readable media. The instructions 1124 may be transmitted or received over a network 1126 via the network interface device 1120.

While computer-readable medium 1122 is shown in an example embodiment to be a single medium, the term “computer-readable medium” should be taken to include a single medium or multiple media (e.g., a centralized or distributed database, or associated caches and servers) able to store instructions (e.g., instructions 1124). The computer-readable medium may include any medium that is capable of storing instructions (e.g., instructions 1124) for execution by the processors (e.g., processors 1102) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### Additional Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any feature mentioned in one claim category, e.g. method, can be claimed in another claim category, e.g. computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject-matter may include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein may be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed by the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

The following applications are incorporated by reference in their entirety for all purposes: (1) U.S. Pat. No. 10,679,729, entitled “Haplotype Phasing Models,” granted on Jun. 9, 2020, (2) U.S. Pat. No. 10,223,498, entitled “Discovering Population Structure from Patterns of Identity-By-Descent,” granted on Mar. 5, 2019, (3) U.S. Pat. No. 10,720,229, entitled “Reducing Error in Predicted Genetic Relationships,” granted on Jul. 21, 2020, (4) U.S. Pat. No. 10,558,930, entitled “Local Genetic Ethnicity Determination System,” granted on Feb. 11, 2020, (5) U.S. Pat. No. 10,114,922, entitled “Identifying Ancestral Relationships Using a Continuous Stream of Input,” granted on Oct. 30, 2018, and (6) U.S. Patent Publication Application No., entitled “Linking Individual Datasets to a Database,” US2021/0216556, published on Jul. 15, 2021.

What is claimed is:

1. A computer-implemented method for identifying one or more segments of a target dataset that match segments of other datasets in a database, the computer-implemented method comprising:
encoding the target dataset to generate a pair of encoded target bitmap sequences based on an encoding scheme, wherein the target dataset comprises a pair of data value sequences, the encoding scheme defines encoding values based on homogeneity between the pair of data value sequences, and the pair of encoded target bitmap sequences comprises a first encoded target bitmap sequence that encodes a first type of homogeneous locations and a second encoded target bitmap sequence that encodes a second type of homogeneous locations;
comparing the pair of encoded target bitmap sequences with other pairs of encoded bitmap sequences to identify homogeneous mismatched locations, the other encoded bitmap sequences generated from the other datasets using the encoding scheme, wherein a homogeneous mismatched location is a location where the target dataset and the other dataset in comparison are both homogeneous but have different types of homogeneity at the location; and
identifying a matched segment between the target dataset and one of the other datasets based on the homogeneous mismatched locations identified, wherein the matched segment is contained within two homogeneous mismatched locations.

2. The computer-implemented method of claim 1, wherein comparing the pair of encoded target bitmap sequences with the other pairs of encoded bitmap sequences to identify homogeneous mismatched locations comprises:
sampling the pair of encoded target bitmap sequences to generate a pair of sparse target bitmap sequences; and
comparing the pair of sparse target bitmap sequences to other pairs of sparse bitmap sequences.

3. The computer-implemented method of claim 2, wherein identifying the matched segment between the target dataset and one of the other datasets based on homogeneous mismatched locations identified comprises:
using the comparison between the pair of sparse target bitmap sequences to other pairs of sparse bitmap sequences as a pre-scan to eliminate mismatches; and
comparing, responsive to one of the other datasets passing the pre-scan, the target dataset and said one of the other datasets to identify the matched segment.

4. The computer-implemented method of claim 2, wherein comparing the pair of encoded target bitmap sequences with one of the other pairs of encoded bitmap sequences to identify the homogeneous mismatched locations further comprises:
identifying a seed range of match between the target dataset and another dataset corresponding to said one of the other pairs of encoded bitmap sequences; and
comparing the pair of sparse target bitmap sequences with one of the other pairs of sparse bitmap sequences upstream and downstream of the seed range to identify the homogeneous mismatched locations.

5. The computer-implemented method of claim 4, wherein comparing the pair of encoded target bitmap sequences with one of the other pairs of encoded bitmap sequences upstream and downstream of the seed range stops at a threshold range.

6. The computer-implemented method of claim 1, wherein identifying a matched segment between the target dataset and one of the other datasets based on the homogeneous mismatched locations identified comprises:
comparing the pair of encoded target bitmap sequences and a pair of encoded bitmap sequences corresponding to said one of the other datasets location-by-location to identify the homogeneous mismatched locations; and
identifying a candidate segment that is between two homogeneous mismatched locations;
determining a length of the candidate segment; and
determining, responsive to the length being larger than a threshold, that the candidate segment is a matched segment. The computer-implemented method of claim 6, wherein the pair of encoded target bitmap sequences are generated from unphased data of the target dataset.

8. The computer-implemented method of claim 1, wherein comparing the pair of encoded target bitmap sequences with another pair of encoded bitmap sequences to identify homogeneous mismatched locations comprise:
comparing the first encoded target bitmap sequence that encodes the first type of homogeneous locations of the target dataset to a second encoded bitmap sequence of said another pair, the second encoded bitmap sequence encoding the second type of homogeneous locations of another dataset; and
identifying a common location that indicates the target dataset and the other dataset in comparison are both homogeneous.

9. The computer-implemented method of claim 8, wherein comparing the first encoded target bitmap sequence that encodes the first type of homogeneous locations of the target dataset to the second encoded bitmap sequence of said another pair comprising running both the first encoded target bitmap sequence of the target dataset and the second encoded bitmap sequence of said another pair through a bitwise AND operation.

10. The computer-implemented method of claim 1, wherein the encoding scheme defines that the first encoded target bitmap sequence has a first value if the pair of data value sequences are homogeneous of the first type and has a second value otherwise, and the encoding scheme defines that the second encoded target bitmap sequence has the first value if the pair of data value sequences are homogeneous of the second type and has the second value otherwise.

11. The computer-implemented method of claim 1, wherein the matched segment is an identity by descent (IBD) segment between two individuals.

12. The computer-implemented method of claim 1, wherein the target dataset corresponds to a target DNA dataset of a target individual and the other datasets correspond to other DNA datasets of other individuals.

13. The computer-implemented method of claim 12, wherein the first type of homogeneous locations corresponds to major alleles, the second type of homogeneous locations corresponds to minor alleles, the pair of data value sequences of the target dataset corresponds to a pair of DNA sequences, and the homogeneity between the pair of data value sequences corresponds to homozygosity between the pair of DNA sequences.

14. A system comprising:
a computing device comprising one or more processors and memory configured to store instructions, wherein the instructions, when executed by the one or more processors, cause the one or more processors to perform steps comprising:
encoding a target dataset to generate a pair of encoded target bitmap sequences based on an encoding scheme, wherein the target dataset comprises a pair of data value sequences, the encoding scheme defines encoding values based on homogeneity between the pair of data value sequences, and the pair of encoded target bitmap sequences comprises a first encoded target bitmap sequence that encodes a first type of homogeneous locations and a second encoded target bitmap sequence that encodes a second type of homogeneous locations;
comparing the pair of encoded target bitmap sequences with other pairs of encoded bitmap sequences to identify homogeneous mismatched locations, the other encoded bitmap sequences generated from the other datasets using the encoding scheme, wherein a homogeneous mismatched location is a location where the target dataset and the other dataset in comparison are both homogeneous but have different types of homogeneity at the location; and
identifying a matched segment between the target dataset and one of the other datasets based on the homogeneous mismatched locations identified, wherein the matched segment is contained within two homogeneous mismatched locations; and
a graphical user interface configured to present result related to the identified matched segment to a user.

15. The system of claim 14, wherein comparing the pair of encoded target bitmap sequences with the other pairs of encoded bitmap sequences to identify homogeneous mismatched locations comprises:
sampling the pair of encoded target bitmap sequences to generate a pair of sparse target bitmap sequences; and
comparing the pair of sparse target bitmap sequences to other pairs of sparse bitmap sequences.

16. The system of claim 15, wherein identifying the matched segment between the target dataset and one of the other datasets based on homogeneous mismatched locations identified comprises:
using the comparison between the pair of sparse target bitmap sequences to other pairs of sparse bitmap sequences as a pre-scan to eliminate mismatches; and
comparing, responsive to one of the other datasets passing the pre-scan, the target dataset and said one of the other datasets to identify the matched segment.

17. The system of claim 15, wherein comparing the pair of encoded target bitmap sequences with one of the other pairs of encoded bitmap sequences to identify the homogeneous mismatched locations further comprises:
identifying a seed range of match between the target dataset and another dataset corresponding to said one of the other pairs of encoded bitmap sequences; and
comparing the pair of sparse target bitmap sequences with one of the other pairs of sparse bitmap sequences upstream and downstream of the seed range to identify the homogeneous mismatched locations.

18. The system of claim 14, wherein comparing the pair of encoded target bitmap sequences with another pair of encoded bitmap sequences to identify homogeneous mismatched locations comprise:
comparing the first encoded target bitmap sequence that encodes the first type of homogeneous locations of the target dataset to a second encoded bitmap sequence of said another pair, the second encoded bitmap sequence encoding the second type of homogeneous locations of another dataset; and
identifying a common location that indicates the target dataset and the other dataset in comparison are both homogeneous.

19. The system of claim 18, wherein comparing the first encoded target bitmap sequence that encodes the first type of homogeneous locations of the target dataset to the second encoded bitmap sequence of said another pair comprising running both the first encoded target bitmap sequence of the target dataset and the second encoded bitmap sequence of said another pair through a bitwise AND operation.

20. A non-transitory computer-readable medium configured to store instructions, wherein the instructions, when executed by one or more processors, cause the one or more processors to perform steps comprising:
encoding a target dataset to generate a pair of encoded target bitmap sequences based on an encoding scheme, wherein the target dataset comprises a pair of data value sequences, the encoding scheme defines encoding values based on homogeneity between the pair of data value sequences, and the pair of encoded target bitmap sequences comprises a first encoded target bitmap sequence that encodes a first type of homogeneous locations and a second encoded target bitmap sequence that encodes a second type of homogeneous locations;
comparing the pair of encoded target bitmap sequences with other pairs of encoded bitmap sequences to identify homogeneous mismatched locations, the other encoded bitmap sequences generated from the other datasets using the encoding scheme, wherein a homogeneous mismatched location is a location where the target dataset and the other dataset in comparison are both homogeneous but have different types of homogeneity at the location; and
identifying a matched segment between the target dataset and one of the other datasets based on the homogeneous mismatched locations identified, wherein the matched segment is contained within two homogeneous mismatched locations.
